SEQUENCE LISTING (26 sequences)

SEQ ID NO: 1 - DNA, 22 bp, Artificial Sequence
  Synthetic primer
tgaccgctacttctccgtgact

SEQ ID NO: 2 - DNA, 20 bp, Artificial Sequence
  Synthetic primer
ccagagcacaaaggaaacca

SEQ ID NO: 3 - DNA, 22 bp, Artificial Sequence
  Synthetic primer
tcacaaaacctctgacctaccc

SEQ ID NO: 4 - DNA, 22 bp, Artificial Sequence
  Synthetic primer
tccacagttctccaccctacaa

SEQ ID NO: 5 - DNA, 23 bp, Artificial Sequence
  Synthetic primer
accatccctcaactccaccaagt

SEQ ID NO: 6 - DNA, 20 bp, Artificial Sequence
  Synthetic primer
ggaaaactgcctccatcgtc

SEQ ID NO: 7 - DNA, 20 bp, Artificial Sequence
  Synthetic primer
tcgctatgagacggtggaaa

SEQ ID NO: 8 - DNA, 20 bp, Artificial Sequence
  Synthetic primer
agcacaaccaatagcccaag

SEQ ID NO: 9 - DNA, 20 bp, Artificial Sequence
  Synthetic primer
gaaagcagcccagacactga

SEQ ID NO: 10 - DNA, 20 bp, Artificial Sequence
  Synthetic primer
agcacaaccaacagcccaag

SEQ ID NO: 11 - DNA, 24 bp, Artificial Sequence
  Synthetic primer
tcggaaaggtgaagtaacttgtcc

SEQ ID NO: 12 - DNA, 20 bp, Artificial Sequence
  Synthetic primer
tccatgggatcaagacacag

SEQ ID NO: 13 - DNA, 22 bp, Artificial Sequence
  Synthetic primer
tgtgatggtgggaatgggtcag

SEQ ID NO: 14 - DNA, 22 bp, Artificial Sequence
  Synthetic primer
tttgatgtcacgcacgatttcc

SEQ ID NO: 15 - DNA, 25 bp, Artificial Sequence
  Synthetic primer
cagaagtggtgatcaagatgcctat

SEQ ID NO: 16 - DNA, 20 bp, Artificial Sequence
  Synthetic primer
gagcttttgggaggctgctt

SEQ ID NO: 17 - DNA, 23 bp, Artificial Sequence
  Synthetic primer
tggagcacaacaagatccagaat

SEQ ID NO: 18 - DNA, 19 bp, Artificial Sequence
  Synthetic primer
cccctgaacgcagttttca

SEQ ID NO: 19 - DNA, 21 bp, Artificial Sequence
  Synthetic primer
ccgctctacctctgtccttca

SEQ ID NO: 20 - DNA, 24 bp, Artificial Sequence
  Synthetic primer
ggtgatctgacttctggtcttgag

SEQ ID NO: 21 - DNA, 22 bp, Artificial Sequence
  Synthetic primer
gtgactgccatcgagatcgtac

SEQ ID NO: 22 - DNA, 20 bp, Artificial Sequence
  Synthetic primer
caaactttcgggccacattg

SEQ ID NO: 23 - DNA, 19 bp, Artificial Sequence
  Synthetic primer
ggcccagagagaacggaac

SEQ ID NO: 24 - DNA, 20 bp, Artificial Sequence
  Synthetic primer
ttcccgttgttgaggtgctt

SEQ ID NO: 25 - DNA, 18 bp, Artificial Sequence
  Synthetic primer
gcatggccttccgtgttc

SEQ ID NO: 26 - DNA, 21 bp, Artificial Sequence
  Synthetic primer
cctgcttcaccaccttcttga

(19) United States
(12) Patent Application Publication
(10) Pub. No.: US 2014/0322242 A1
(43) Pub. Date: Oct. 30, 2014

(54) MATERIALS AND METHODS FOR THE PREVENTION AND TREATMENT OF CANCER

(71) Applicants: Paul S. Frenette, New York, NY (US); Claire Magnon, Saint Cloud (FR)

(72) Inventors: Paul S. Frenette, New York, NY (US); Claire Magnon, Saint Cloud (FR)

(73) Assignee: Icahn School of Medicine at Mount Sinai, New York, NY (US)

(21) Appl. No.: 13/991,091

(22) PCT Filed: Nov. 24, 2011
(86) PCT No.: PCT/US11/62147

§ 371 Date: Mar. 26, 2014

Related U.S. Application Data
(60) Provisional application No. 61/417,162, filed on Nov. 24, 2010.

(51) Int. Cl. C07K 16/30; A61K 45/06; A61K 31/5375; A61K 31/5513; C12Q 1/68; A61K 31/4453; A61K 31/439; A61K 31/46; G01N 33/574; A61K 39/395; A61K 38/17

(52) U.S. Cl. CPC C07K 16/30; A61K 39/3955; A61K 45/06; A61K 31/5375; A61K 31/5513; A61K 38/1703; A61K 31/4453; A61K 31/439; A61K 31/46; G01N 33/57434; C12Q 1/6886
USPC 424/174.1; 514/230.5; 514/220; 514/17.4; 514/317; 514/291; 514/304; 435/7.1; 435/6.12

(57) ABSTRACT

Methods of treating or preventing cancer, or ameliorating a symptom thereof, by administering a muscarinic receptor inhibitor to inhibit cancer metastasis and/or a β adrenergic receptor inhibitor to inhibit tumor initiation are provided. Also provided are pharmaceutical compositions for treating or preventing cancer comprising a muscarinic receptor inhibitor and/or a β adrenergic receptor inhibitor.

This invention was made with government support under grant number W81XWH-07-1-0165 awarded by the Department of Defense. The government has certain rights in the invention.

### FIELD OF THE INVENTION

The disclosure relates to methods of treating cancer by administering muscarinic receptor inhibitors and/or β adrenergic receptor inhibitors. Also provided are pharmaceutical compositions useful for treating cancer comprising muscarinic receptor inhibitors and β adrenergic receptor inhibitors.

### BACKGROUND

Cancer is one of the most significant health scourges worldwide and in the U.S., when considered from the perspective of lives lost or compromised as well as resources consumed in the fight against it. In the U.S., about 1,529,560 new cancer cases are expected to be diagnosed and 569,490 Americans are expected to die of cancer in 2010. Cancer is the second most common cause of death in the U.S., exceeded only by heart disease. The National Institutes of Health estimate overall costs of cancer in 2010 at $263.8 billion: $102.8 billion for direct medical costs (total of all health expenditures); $20.9 billion for indirect morbidity costs (cost of lost productivity due to illness); and $140.1 billion for indirect mortality costs (cost of lost productivity due to premature death).

Studies have indicated that the neural environment contributes to malignant tumor progression. For example, conditions producing a high degree of chronic or intermittent stress, a neural phenomenon, have been reported to increase tumor incidence, tumor size, and the incidence of metastasis. In addition, tumor growth is enhanced in mice subjected to a single inescapable electric shock. Clinical evidence indicates that perineural invasion by human prostate tumors correlates with poor prognosis. Conversely, spinal cord injury patients exhibited a lower prevalence of prostate cancer associated with a reduced risk of prostate cancer. The presence and function of nerves and the neural environment in tumor development and progression has remained unclear, however.

Beta adrenergic and muscarinic receptors are two types of receptor proteins involved in neural transmissions, with β-adrenergic receptors (β1, β2, and β3) generally involved in sympathetic nervous system (SNS) transmission and muscarinic receptors (M1-M4), e.g., the M1R (Chrm1) muscarinic receptor, generally involved in parasympathetic nervous system transmission. More particularly, β adrenergic receptors, or β adrenoceptors, are G protein-coupled receptors with a subset of catecholamines (norepinephrine, epinephrine, isoprenaline, but not dopamine) as ligands. The β adrenergic receptors are involved in the SNS-mediated flight-or-fight response. Recently, Palm et al., Int. J. Cancer 118:2744-2749 (2006) disclosed the use of propranolol to treat metastatic cancer, but not cancer initiation, in a heterotopic mouse model. Propranolol is a non-selective-adrenoceptor inhibitor. Muscarinic receptors are G protein-coupled acetylcholine receptors that have several functions, including recovery from neural firing. An antagonist of a muscarinic receptor was recently reported to have beneficial effect in treating hot flashes in men resulting from combined radiation and androgen deprivation therapy for advanced prostate cancer. U.S. Pat. Publication No. 2007/0281997. The publication disclosed that muscarinic receptor antagonists were effective in treating hot flashes, but did not address any effect on the cancer itself, or its metastasis.

In view of the foregoing observations, it is apparent that there is significant neural involvement in cancer development, and there remains a need in the art for therapeutics, and methods of using such therapeutics, that are effective in treating, preventing, or ameliorating a symptom associated with cancer engraftment and/or metastasis by affecting neural function.

### SUMMARY

The present disclosure provides an economical approach to the treatment of a variety of cancers by affecting the physiology of the neural environment that is, or is reasonably expected to become, associated with cancer. More particularly, the disclosure provides at least one inhibitor of a β-adrenergic receptor and/or at least one inhibitor of a muscarinic receptor in a variety of forms for administration to a subject (e.g., a human) exhibiting, or at risk of exhibiting, a tumor-forming cancer.

In one aspect of the disclosure, there is provided a method of treating cancer comprising administering to a cancer patient a therapeutically effective amount of a muscarinic receptor inhibitor. Viewed alternatively, the aspect provides for use of a therapeutically or prophylactically effective amount of a muscarinic receptor inhibitor in the preparation of a medicament for the treatment of cancer. In some embodiments, the method or use further comprises administering a β adrenergic receptor inhibitor. In some embodiments, the adrenergic receptor inhibitor is a β2 adrenergic receptor inhibitor, a β3 adrenergic receptor inhibitor, or both a β2 adrenergic receptor inhibitor and a β3 adrenergic receptor inhibitor. In some embodiments, the β2 adrenergic receptor inhibitor is selected from the group consisting of an anti-β2 antibody, butaxamine, propranolol, and ICI-118,551. In some embodiments, the β3 adrenergic receptor inhibitor is selected from the group consisting of an anti-β3 antibody, and SR 59230A. In some embodiments, the β adrenergic receptor inhibitor is a nonspecific β adrenergic receptor inhibitor. In some embodiments, the nonspecific β adrenergic receptor inhibitor is selected from the group consisting of alprenolol, bucindolol, carteolol, carvedilol, labetalol, nadolol, penbutolol, pindolol, sotalol, and timolol. In some embodiments, the adrenergic receptor inhibitor is administered in an amount effective to inhibit tumor initiation. In some embodiments, the muscarinic receptor inhibitor is an M1 receptor inhibitor or the muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide and telenzepine. In some embodiments of the method, the muscarinic receptor inhibitor is administered in an amount effective to inhibit tumor metastasis. In some embodiments, the patient is a woman or non-human animal, such as a non-human female. In some embodiments, the patient is not undergoing androgen deprivation therapy and/or is not undergoing radiation therapy. In some embodiments, the patient is not in need of treatment for, or is not experiencing, hot flashes associated with androgen deprivation therapy.

Methods and uses according to the disclosure contemplate prevention, treatment, or amelioration of a symptom associated with any tumor-forming cancer, including a cancer selected from the group consisting of prostate cancer, breast cancer, melanoma, gastric cancer, colon cancer, liver cancer, pancreatic cancer, esophageal cancer, lung cancer, and urogenital cancers.

The inhibitor is administered by any route known in the art. The inhibitor is administered at a dosage sufficient to achieve a desired therapeutic or prophylactic effect and is determined on a case-by-case basis in view of a number of considerations known in the art, e.g., weight, age, general health, diet, and the like. In some embodiments, the inhibitor is administered at a daily dosage of 10 μg/kg to 10 mg/kg.

In some embodiments, the method or use further comprises administering a therapeutically effective amount of a chemotherapeutic agent. Exemplary chemotherapeutic agents are 5-fluorouracil (5-FU), azathioprine, cyclophosphamide, antimetabolites (such as fludarabine), etoposide, doxorubicin, methotrexate, vincristine, carboplatin, cis-platinum and the taxanes (such as taxol), monoclonal antibodies such as Avastin or Herceptin, and growth pathway inhibitors such as gleevac.

In methods according to the disclosure, the muscarinic receptor inhibitor and adrenergic receptor inhibitor are administered immediately before, during, or immediately after cancer surgery.

Another aspect of the disclosure is drawn to a method of inhibiting tumor initiation comprising administering to a patient in need thereof a therapeutically effective amount of a β2 adrenergic receptor inhibitor and a β3 adrenergic receptor inhibitor. In the alternative, this aspect provides for use of a therapeutically effective amount of a β2 adrenergic receptor inhibitor and a β3 adrenergic receptor inhibitor in the preparation of a medicament for inhibiting tumor initiation. In various embodiments, the β2 adrenergic receptor inhibitor is selected from the group consisting of an anti-β2 antibody, butaxamine, propranolol and ICI-118,551. In some embodiments, the β3 adrenergic receptor inhibitor is selected from the group consisting of an anti-β3 antibody and SR 59230A. In various embodiments, the adrenergic receptor inhibitor is administered immediately before, during, or immediately after cancer surgery. In some embodiments of the method, the cancer is selected from the group consisting of prostate cancer, breast cancer, melanoma, colon cancer, gastric cancer, liver cancer, pancreatic cancer, esophageal cancer, lung cancer, and urogenital cancers.

The adrenergic receptor inhibitor is administered by any route known in the art. The inhibitor is administered at a dosage sufficient to achieve a desired therapeutic or prophylactic effect and is determined on a case-by-case basis in view of a number of considerations known in the art (e.g., weight, age, general health, diet, and the like. In some embodiments, the inhibitor is administered at a daily dosage of 10 μg/kg to 10 mg/kg.

Another aspect of the disclosure is directed to a method of inhibiting tumor cell metastasis comprising administering to a patient in need thereof a therapeutically effective amount of a muscarinic receptor inhibitor. In some embodiments, the muscarinic receptor inhibitor is an M1 receptor inhibitor. In various embodiments of the method, the muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, and telenzepine. It should be noted that the muscarinic receptor inhibitor is administered immediately before, during, or immediately after cancer surgery. As for aspects described above, the cancer is selected from the group consisting of prostate cancer, breast cancer, melanoma, colon cancer, and gastric cancer. In some embodiments, the muscarinic receptor inhibitor is administered by any route known in the art. The inhibitor is administered at a dosage sufficient to achieve a desired therapeutic or prophylactic effect and is determined on a case-by-case basis in view of a number of considerations known in the art (e.g., weight, age, general health, diet, and the like. In some embodiments, the inhibitor is administered at a daily dosage of 10 μg/kg to 10 mg/kg.

Another aspect of the disclosure relates to a pharmaceutical composition for treating cancer comprising a muscarinic receptor inhibitor and an adrenergic receptor inhibitor. In some embodiments, the composition further comprises a pharmaceutically acceptable carrier. In various embodiments, the muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, and telenzepine, and the adrenergic receptor inhibitor is selected from the group consisting of an anti-β2 antibody, an anti-β3 antibody, butoxamine, propranolol, SR 59230A, and ICI-118,551. In some embodiments, the composition further comprises a chemotherapeutic agent.

Another aspect of the disclosure relates to a method of inhibiting tumor cell metastasis in a subject comprising administering a prophylactically or therapeutically effective amount of a muscarinic receptor inhibitor. Alternatively, the aspect provides for use of a therapeutically effective amount of a muscarinic receptor inhibitor in the preparation of a medicament for inhibiting tumor cell metastasis in a patient. In some embodiments, the muscarinic receptor inhibitor is an M1 receptor inhibitor, or is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide and telenzepine. In some embodiments, the cancer is selected from the group consisting of prostate cancer, breast cancer, melanoma, colon cancer, gastric cancer, liver cancer, pancreatic cancer, esophageal cancer, lung cancer and urogenital cancers. In some embodiments, the muscarinic receptor inhibitor is administered at a daily dosage of 10 μg/kg to 10 mg/kg. In some embodiments, the subject is a human or a non-human animal such as a non-human mammal.

Another aspect of the disclosure is a method for treating cancer in a subject comprising administering a prophylactically or therapeutically effective amount of an inhibitor of NF-L+ neo-fiber development. Alternatively, this aspect provides a use for use of a prophylactically or therapeutically effective amount of an inhibitor of NF-L+ neo-fiber development in the preparation of a medicament for treating cancer in a subject. In some embodiments, the inhibitor is an inhibitor of G-CSF.

Another aspect of the disclosure is a method of diagnosing cancer comprising detecting a neurofilament-L marker in a tissue subject to diagnostic cancer assay. In some embodiments, the tissue is prostate. A related aspect of the disclosure is drawn to the method further comprising identifying the tissue as exhibiting an absence of a marker for neurofilament H.

Another aspect of the disclosure is a method for inhibiting cancer initiation in a tissue of a subject comprising administering a prophylactically or therapeutically effective amount of a sympathetic nervous system inhibitor. Alternatively, the aspect is drawn to use of a prophylactically or therapeutically effective amount of a sympathetic nervous system inhibitor in the preparation of a medicament for inhibiting cancer initiation in a tissue of a subject. In some embodiments, the tissue is prostate. In some embodiments, the subject is a human or non-human animal, such as a non-human mammal.

Another aspect of the disclosure is a method for treating cancer in a subject comprising administering a prophylactically or therapeutically effective amount of an inhibitor of the parasympathetic nervous system. Alternatively, the aspect can be understood as drawn to use of a prophylactically or therapeutically effective amount of an inhibitor of the parasympathetic nervous system in the preparation of a medicament for treating cancer in a subject. In some embodiments, treating cancer comprises inhibiting metastasis. In some embodiments, the inhibitor is a Chrm1 inhibitor, carbamoylcholine (carbachol) or pirenzepine (PZP).

Another aspect of the disclosure is a method for treating cancer comprising administering a prophylactically or therapeutically effective amount of a β adrenergic receptor inhibitor in combination with a second anti-cancer therapeutic. Alternatively, the aspect is drawn to use of a β adrenergic receptor inhibitor in combination with a second anti-cancer therapeutic in the preparation of a medicament for the treatment of cancer. In some embodiments, the second anti-cancer therapeutic is a type-1 muscarinic receptor inhibitor.

Particular aspects of the disclosure are described in the following enumerated paragraphs.

1. Use of a therapeutically or prophylactically effective amount of a muscarinic receptor inhibitor in the preparation of a medicament for the treatment of cancer.

2. The use of paragraph 1 further comprising administering a therapeutically effective amount of a β adrenergic receptor inhibitor.

3. The use of paragraph 2 wherein the adrenergic receptor inhibitor is a β2 adrenergic receptor inhibitor, a β3 adrenergic receptor inhibitor, or both a β2 adrenergic receptor inhibitor and a β3 adrenergic receptor inhibitor.

4. The use of paragraph 3 wherein the β2 adrenergic receptor inhibitor is selected from the group consisting of an anti-β2 antibody, butaxamine, propranolol and ICI-118,551.

5. The use of paragraph 3 wherein the β3 adrenergic receptor inhibitor is selected from the group consisting of an anti-β3 antibody and SR 59230A.

6. The use of any one of paragraphs 2 to 5 wherein the β adrenergic receptor inhibitor is administered in an amount effective to inhibit tumor initiation.

7. The use of any one of the above paragraphs wherein the muscarinic receptor inhibitor is an M1 receptor inhibitor.

8. The use of any one of the above paragraphs wherein the muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide and telenzepine.

9. The use of any one of paragraphs 1 to 8 wherein the muscarinic receptor inhibitor is administered in an amount effective to inhibit tumor metastasis.

10. The use of any one of the above paragraphs wherein the cancer is selected from the group consisting of prostate cancer, breast cancer, melanoma, gastric cancer, colon cancer, liver cancer, pancreatic cancer, esophageal cancer, lung cancer and urogenital cancers.

11. The use of any one of the above paragraphs wherein the inhibitor is administered at a daily dosage of 10 μg/kg to 10 mg/kg.

12. The use of any one of the above paragraphs further comprising administering a therapeutically effective amount of a chemotherapeutic agent.

13. Use of a therapeutically effective amount of a β2 adrenergic receptor inhibitor and a β3 adrenergic receptor inhibitor in the preparation of a medicament for inhibiting tumor initiation.

14. The use of paragraph 13 wherein the β2 adrenergic receptor inhibitor is selected from the group consisting of an anti-β2 antibody, butaxamine, propranolol and ICI-118,551.

15. The use of paragraph 13 wherein the β3 adrenergic receptor inhibitor is selected from the group consisting of an anti-β3 antibody and SR 59230A.

16. The use of any one of paragraphs 13 to 15 wherein the cancer is selected from the group consisting of prostate cancer, breast cancer, melanoma, colon cancer, gastric cancer, liver cancer, pancreatic cancer, esophageal cancer, lung cancer and urogenital cancers.

17. The use of any one paragraphs 13 to 16 wherein the inhibitor is administered at a daily dosage of 10 μg/kg to 10 mg/kg.

18. Use of a therapeutically effective amount of a muscarinic receptor inhibitor in the preparation of a medicament for inhibiting tumor cell metastasis in a patient.

19. The use of paragraph 18 wherein the muscarinic receptor inhibitor is an M1 receptor inhibitor.

20. The use of paragraph 18 wherein the muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide and telenzepine.

21. The use of any one of paragraphs 18 to 20 wherein the cancer is selected from the group consisting of prostate cancer, breast cancer, melanoma, colon cancer, gastric cancer, liver cancer, pancreatic cancer, esophageal cancer, lung cancer and urogenital cancers.

22. The use of any one of paragraphs 18 to 21 wherein the muscarinic receptor inhibitor is administered at a daily dosage of 10 μg/kg to 10 mg/kg.

23. A pharmaceutical composition for treating cancer comprising a muscarinic receptor inhibitor and a β adrenergic receptor inhibitor.

24. The composition of paragraph 23 further comprising a pharmaceutically acceptable carrier.

25. The composition of paragraph 23 or 24 wherein the muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide and, telenzepine and the β adrenergic receptor inhibitor is selected from the group consisting of an anti-β2 antibody, an anti-β3 antibody, butoxamine, propranolol, SR 59230A, alprenolol, bucindolol, carteolol, carvedilol, labetalol, nadolol, penbutolol, pindolol, sotalol, timolol and ICI-118,551.

26. Use of a prophylactically or therapeutically effective amount of an inhibitor of NF-L+ neo-fiber development in the preparation of a medicament for treating cancer in a subject.

27. The use of paragraph 26 wherein the inhibitor is an inhibitor of G-CSF.

28. A method of diagnosing cancer comprising detecting a neurofilament-L marker in a tissue subject to diagnostic cancer assay.

29. The method of paragraph 28 wherein the tissue is prostate.

30. The method of paragraph 28 further comprising identifying the tissue as exhibiting an absence of a marker for neurofilament H.

31. Use of a prophylactically or therapeutically effective amount of a sympathetic nervous system inhibitor in the preparation of a medicament for inhibiting cancer initiation in a tissue of a subject.

32. The use of paragraph 31 wherein the tissue is prostate.

33. The use of paragraph 31 wherein the subject is a human.

34. Use of a therapeutically effective amount of an inhibitor of the parasympathetic nervous system in the preparation of a medicament for treating cancer in a subject.

35. The use of paragraph 34 wherein treating cancer comprises inhibiting metastasis.

36. The use of paragraph 34 wherein the inhibitor is a Chrm1 inhibitor, carbamoylcholine (carbachol) or pirenzepine (PZP).

37. Use of a β adrenergic receptor inhibitor in combination with a second anti-cancer therapeutic in the preparation of a medicament for the treatment of cancer.

38. The use of paragraph 37 wherein the second anti-cancer therapeutic is a type-1 muscarinic receptor inhibitor.

Other features and advantages of the present disclosure will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1. Tumor initiation and metastasis are regulated by a dual neural mechanism. Sympathetic fibers deliver adrenergic signals (Noradrenaline, NA) that control the initial development of the tumor through the β2 and β3 adrenergic receptors (AR). Parasympathetic innervation also invades tumors, delivering acetylcholine (Ach) that promotes tumor cell proliferation and egress to lymph nodes and distant organs through the type-1 muscarinic receptor (Chrm1) in the stroma. G-CSF protects SNS fibers from reactive oxygen species (ROS)-induced damage (e.g., by 6-hydroxydopamine; not shown) and promotes the development of new PNS fibers in the tumor, increasing the cholinergic tone, tumor cell proliferation and invasion. Sympathetic nervous system (SNS), Parasympathetic nervous system (PNS).

FIG. 2. Granulocyte-Colony Stimulating Factor- (G-CSF-) mediated invasion and metastasis are correlated with cholinergic tumor neo-nerve fiber formation. a-b, In vivo bioluminescence imaging of Balb/c nu/nu males harboring PC-3luc cells injected orthotopically into the ventral prostate, 11 weeks after graft. a, Without development of metastasis. b, With distant metastases. c, Ex vivo imaging of the primary tumor. d-e, Histological confirmation of tumor tissues from the primary (d), and a lung metastasis (e). f-h, Ex vivo bioluminescence of metastases within the colon (f), liver (g), and lung (h) upon sacrifice at week 11. i, Serial quantification of bioluminescence intensities in primary tumors between weeks 1 and 11 after grafting. Chemical sympathetic nervous system (SNS) denervation with 6-hydroxydopamine (6OHDA) markedly inhibited tumor initiation and G-CSF administration rescued the development of primary tumors initially sympathectomized with 6OHDA. j, G-CSF induced invasion of inguinal lymph nodes and metastases, but did not induce metastases in 6OHDA-lesioned animals. k, Quantification of neural area per tumor field (LUMPlanFI 60× NA 0.90 ∞ objective, five fields per animal) within sections of tumor prostates at 11 weeks. Note that G-CSF alone stimulated the sprouting of cholinergic neo-fibers (NF-L+/VAChT+), but in combination with 6OHDA, G-CSF protected sympathetic TH+ fibers and reduced parasympathetic VAChT+ nerve density. One field=0.01 mm². Results are shown as mean±SEM. Balb/c nu/nu males, PBS, n=11; G-CSF, n=14, 6OHDA, n=8, 6OHDA+G-CSF, n=11. l, Representative immunofluorescence staining of NF-L, NF-H, TH and VAChT tumour nerves in red (DAPI, blue). m, Timeline for cMyc-induced tumor progression and therapeutic schedule for G-CSF injections. Illustrative examples of hematoxylin- and eosin-stained prostate sections from four-month-old cMyc transgenic mice: normal prostate acinus, mouse prostate intraepithelial neoplasia (mPIN) delineated by the fibromuscular stroma, and invasive cancer zone. n, Percentage of mPIN and number of invasive cancer zones per prostate section of G-CSF-treated mice compared to saline group (data obtained from 10 sections per animal, n=3-4 mice). o, Representative images of immunofluorescence staining of NF-L and VAChT tumor nerves of cMyc+ prostate sections from saline- or G-CSF-treated animals (Nerve markers, red, DAPI, blue). *, P<0.05; **, P<0.01; ***, p<0.001; Scale bars, 10 μm.

FIG. 3. G-CSF induces tumor cell proliferation in vivo, but not in vitro. a, Mice treated with G-CSF exhibited a higher proliferative index (PI) for PC-3luc tumor cells orthotopically implanted into the prostate gland than the PI for such cells in tumors from mice previously denervated using 6OHDA. Data were derived from four random fields/section, n=3-4 mice per group. b, Immunofluorescence detection of proliferative PC-3luc cells stained with anti-Ki67 antibody. Scale bar, 50 μm. c, RT-PCR analyses of Granulocyte-Colony Stimulating Factor Receptor (G-CSFR) expression in PC-3luc cells in vitro and in vivo. RNAs were extracted from a PC-3luc primary tumor or a metastasis. RNA extracted from human leukocytes was used as positive control. G-CSFR was not expressed in PC-3luc cells in vitro and in vivo. ACTB, β-actin control. d, G-CSF did not induce the proliferation of PC-3luc cells in vitro compared with that of human promyelocytic leukemia cells (HL-60) or human leukemic monocyte lymphoma cells (U937). *, P<0.05, **, P<0.01 compared to control group (Mann & Whitney test). Error bars indicate standard error.

FIG. 4. G-CSF-mediated cholinergic nerve sprouting in the healthy prostate is associated with the proliferation of epithelial cells. a, At steady state (upper panels), adrenergic varicose fibers stained for tyrosine hydroxylase (TH) lie in the stromal compartment beneath the epithelium. In contrast, cholinergic fibers expressing the vesicular acetylcholine transporter (VAChT) tightly surround epithelial cells forming close bonds with smooth muscle cells and the epithelium. These are bona fide nerve fibers because they also express the neuron-specific cytoskeletal subunits of neurofilament-L (NF-L) and/or neurofilament-H(NF-H). Three weeks after the last injection of G-CSF (middle panel), sprouting of NF-L+/VAChT+ cholinergic neo-fibers was stimulated and the network of mature NF-H+ fibers was consolidated. Note that combination of 6OHDA+G-CSF significantly reduced the number of VAChT+ fibers (lower panel). Similar observations were made in tumor tissues (see FIG. 2k). b, Quantification of periacinus nerve areas positive for NF-L, NF-H, TH and VAChT three weeks after the last injection of G-CSF (five fields/section, field surface=0.01 mm2, n=3/group). c, G-CSF treatment also led to proliferation of healthy epithelial cells as determined by anti-Ki67 antibody staining. In contrast, chemical denervation with 6OHDA reduced G-CSF-induced epithelial cell proliferation. d, Illustrative examples of prostate sections showing Ki67+ epithelial cells. *, P<0.05, ***, P<0.001. Scale bars, 50 μm; DAPI, blue. Error bars indicate standard error.

FIG. 5. Chemical sympathetic denervation by 6OHDA induces epithelial cell death in vivo. a-b, 6OHDA damaged specifically TH+ (red) catecholaminergic fibers, four days after the last injection, without any detectable changes on cholinergic fibers (VAChT+, right panels). c-d, 6OHDA triggered prostate epithelial cell death in vivo in healthy (FIG. 5c) or PC-3luc-grafted (FIG. 5d) prostate. Tissues were harvested three weeks after the last injection of G-CSF. In the case of grafted prostate (FIG. 5d), tissues were obtained three weeks after the second round of G-CSF, 11 weeks after grafting. NT, no tumor formed. Data were obtained from 5 fields per section from field surface=0.038 mm², n=2-3 mice. e, Illustrative examples of TUNEL staining in prostate from 6OHDA- or PBS-treated mice. DAPI, blue. f, 6OHDA did not induce any direct cytotoxicity in vitro on cultured PC-3luc cells. **, P<0.01; ***, P<0.001. Error bars indicate standard error.

FIG. 6. Sympathetic nervous system regulates tumor initiation. a-b, Real time quantification of bioluminescence from PC-3luc tumor cells implanted orthotopically into surgically sympathectomized (denervated of hypogastric nerves (HGNx; n=5)) or sham-operated (n=8). b, Immunofluorescence analysis showing sympathectomy as determined by TH and DAPI staining. c-d, Ex vivo quantification of bioluminescence in primary tumors (c), and in lymph nodes and distant metastases (d). e, Timeline for c-Myc-induced tumor progression and therapeutic schedule. Illustrative examples of hematoxylin- and eosin-stained prostate sections from four-month-old c-Myc transgenic mice: normal prostate acinus, mouse prostate intraepithelial neoplasia (mPIN) delineated by the fibromuscular stroma, and invasive cancer zone. f-g, Effect of systemic (6OHDA, n=26) or local HGNx (n=11) denervation of the SNS on the prevalence of mPIN (f) or invasive cancer zones (g) in cMyc transgenic mice. Denervation at 1 month, but not later, significantly reduced the percentage of mPIN. Data were analysed from 10 sections/animal. h, Representative images of hematoxylin and eosin-stained prostate sections from saline- or 6OHDA-treated animals and their respective sympathetic innervation (TH+). The tumor-free zone is seen after treatment with 6OHDA. Note that normal acini are innervated by rare sympathetic sprouts (Box 2) compared with high-grade mPIN areas that are surrounded and infiltrated by dense and thick TH+ fibers (Box 1). Interestingly, mPIN areas in 6OHDA-treated mice are associated with dense TH+ SNS fibers (Box 3). Results are shown as mean±SEM. *, P<0.05; **, P<0.01; ***, p<0.001. Scale bars, 50 μm (e), 1000 μm (h, left and middle images), 200 μm (h, right images). i, Immunofluorescence analysis showing sympathectomy as determined by TH staining in red (DAPI, blue). j, In vivo bioluminescence quantification of LNCaPluc cells orthotopically implanted in the ventral prostate of nu/nu Adrβ2^{-/-} Adrβ3^{-/-} mice (n=8) or nu/nu Adrβ2^{+/+}Adrβ3^{+/+} control littermates (n=8). k, Illustrative TUNEL staining for apoptotic cells in mPIN from a surgically sympathectomized mouse, and quantification of apoptotic cells revealed significant higher numbers of TUNEL+ cells in HGNx mice compared to control (C). Data obtained from 5 fields/animal, field area=0.038 mm², n=6.

FIG. 7. Expression of muscarinic receptors in the normal prostate and cancer cell lines. a, Real-time quantitative PCR analyses of mRNA extracts from prostate cancer cell lines and healthy prostate tissues. The prostate gland predominantly expresses Chrm1 whereas PC-3 tumor cells mostly express Chrm3R. b, Chrm1 expression tended to increase in the prostate gland three weeks after the last injection of G-CSF. Results are shown as mean±SEM.

FIG. 8. Tumor cell proliferation and motility are controlled by Chrm1 (M1R). a-b, Ex vivo quantification of bioluminescence from PC-3luc cells in the prostate gland of Balb/c nu/nu mice in the primary tumor or lymph nodes (a), or in distant organs (b) at week 5. Activation of muscarinic receptors with the cholinomimetic carbachol enhanced lymph node invasion, depopulated primary tumors, and dramatically increased metastatic activity only five weeks post-grafting. Specific blockade of Chrm1 (M1R) with pirenzepine (PZP) inhibited the dissemination of tumor cells towards lymph nodes and the periphery. Balb/c nu/nu males; PBS (n=9), carbachol (n=7), scopolamine+carbachol (n=4), PZP (n=9), PZP+carbachol (n=6). c, Representative images of bioluminescent signal in primary tumors, inguinal lymph nodes and colon metastases from the different treated mouse backgrounds described above. d-e, Inhibition of lymph node invasion (d), and metastases (e) six weeks after orthotopic implantation of PC-3luc cells into carbachol-treated Chrm1^{-/-} nu/nu mice (n=9) compared to Chrm1^{+/-} nu/nu and Chrm1^{+/+} nu/nu controls (n=11) of the same background. In contrast, saline-injected Chrm1^{+/-} nu/nu or Chrm1^{+/+} nu/nu (n=7) and Chrm1^{-/-} nu/nu (n=7) do not display any metastases at this time. f, Serial analyses of the engraftment of PC-3luc cells within Chrm1^{-/-} nu/nu mice (n=7) by comparison to Chrm1^{+/+} nu/nu (n=7) controls. Note that Chrm1 (M1R) disruption does not inhibit tumor initiation by comparison with β2 and β3 adrenergic receptor deficiency in the prostate (see FIG. 2b). g, Representative images of bioluminescent signal from the primary tumors and colon metastases. i-o, Role of Chrm1 (M1R) in prostate cancer progression in transgenic mice. Timeline for c-Myc-induced tumor progression and therapeutic schedule (i). Section of the entire prostate from an animal treated with carbachol, with boxed areas showing higher magnifications of the invasive zone (j-l). m, Percentage of neoplastic acini. n, Number of invasive zones per prostate section is increased after treatment by carbachol (group 2) which mimics the cholinergic input inside the tumor. This effect is prevented by injection of a selective Chrm1 (M1R) antagonist, pirenzepine (group 5). o, Quantification of Ki-67 staining showing that carbachol-induced tumor dissemination is associated with the proliferation of epithelial cells in c-Myc transgenic mice. Results are shown as mean±SEM. *, P<0.05; **, P<0.01; ***, p<0.001. Scale bars 1000 μm (l), 100 μm (j, k). p, Representative images of mice from (q) at different time points. Note that carbachol-treated animals exhibited extensive metastases which were prevented by pharmacologic or genetic disruption of Chrm1 (M1R). q, Kaplan-Meier curves depicting the survival of metastatic nu/nu Chrm1^{+/+} mice treated with carbachol (n=14) by comparison to non-metastatic carbachol-treated nu/nu Chrm1^{-/-} animals (n=6; p=0.0005, log rank test) or nu/nu Chrm1^{+/+} mice treated by PZP+carbachol (n=9; p=0.03, log rank test). r, Histological analyses of prostate chimeric tissues shows a key role for stromal Chrm1. cMyc+ Chrm1^{-/-} prostate tissues were grafted in the dorsal lobe of nu/nu prostate glands (left panel). Tissues from carbachol− (upper panels) or carbachol+PZP-treated (lower panels) mice were harvested 10 weeks after surgery. Hematoxylin- and eosin-stained sections of cMyc+ Chrm1^{-/-} grafts revealed invasive cancer zones (panel 1) and invasive PIN (2) in the carbachol-treated group, but not when Chrm1 was blocked by PZP (panels 4, 5). Immunofluorescence staining for laminin-α2 (light blue) confirms disruption of basement membranes (panels 2, 3) and Ki-67+ (red) proliferative neoplastic epithelial cells (3) in carbachol-treated chimeras, but not in the carbachol+PZP-treated group (panels 5, 6). DAPI, dark blue). *, P<0.05; **, P<0.01; ***, p<0.001. Scale bars 100 μm (j, o), 200 μm (k). Error bars indicate standard error. s-t, Role of Chrm1 in prostate cancer progression in cMyc transgenic mice. Time line for cMyc-induced tumor progression and therapeutic schedule (i). Section of the prostate from an animal treated by carbachol with boxed area showing higher magnification of the invasive zone.

FIG. 9. Cholinergic signals activate proliferation of prostate tumor cells in vivo but not in vitro. a, Incubation of PC-3luc cells in vitro with carbachol does not induce tumor cell proliferation. b, In contrast, carbachol injections induced the proliferation of PC-3luc cells orthotopically implanted into the prostate, as determined by the expression of Ki67. Administration of pirenzepine (PZP), a selective antagonist of the type-1 muscarinic receptor (Chrm1), specifically blocked carbachol-induced tumor cell proliferation. Results are shown as the mean±SEM. Data obtained from five random fields/section, field surface=0.01 mm2. n=3 mice per group; **, P<0.01. Error bars indicate standard error.

FIG. 10. Human invasive prostate adenocarcinoma recruits neo-nerves. a, Illustrative example of hematoxylin- and eosin-stained sections from healthy normal prostate, benign prostatic hyperplasia (BPH), prostatic intraepithelial neoplasia (PIN) and extensive adenocarcinoma. Black arrow indicates normal epithelium. b-d, Immunofluorescence analyses of autonomic innervation of healthy prostate acini (b), high grade prostate PIN (c) and invasive adenocarcinoma (d). Prostate adenocarcinoma developed a thick disorganized neo-neural network (NF-L+) comprising both cholinergic (VAChT+) and adrenergic (TH+) fibers. In contrast, BPH stroma (d) is innervated by discrete VAChT+ fibers and some sparse TH+ sprouts, but is devoid of immature NF-L+ nerves. e, Quantification of immunostained neural areas in human tumor tissues. n=3 cancer samples: Gleason 4+3, cancer invading 50% of the gland but confined {pT2cN0}, with focal extracapsular extension and negative margins {pT3aN0}, or extracapsular extension with involvement of a seminal vesicle {pT3bN0}) and n=3 BPH samples. Data obtained from 5-6 random fields/section, field surface=0.01 mm². Scale bars, 50 μm. ***p<0.001 BPH versus adenocarcinoma for each marker.

FIG. 11. Stromal innervation of normal prostate tissues. Immunofluorescence staining shows faint staining for neuron-specific cytoskeletal neurofilaments (NF-L and NF-H) in stromal areas of the human prostate gland.

FIG. 12. G-CSF stimulates axon total outgrowth of murine fetal neurons in vitro. Neuronal cultures were prepared from the neocortex of embryonic day 15 mouse fetuses as described herein. Neurite outgrowth was assessed by staining neuronal cells with anti-microtubule-associated protein 2 (MAP2) and anti-phosphorylated neurofilament-H (pNF-H), which largely segregate into dendritic and axonal domains, respectively. a, Immunofluorescence analysis showing specific staining for Csfr3 expression on a representative fetal neuron. Control staining was performed with a mixture of mouse anti-pNF-H and MAP2 antibodies followed by an Alexafluor568-conjugated goat anti-rabbit antibody used as secondary antibody for Csfr3 detection. Scale bars, 10 μm. b, Quantification of neurite outgrowth using the “Neurite Outgrowth” module of Metamorph software. Data were analyzed objectively from 40 random fields per growth condition, field surface=0.6 mm². Similar results were obtained from two independent experiments. c, Representative immunofluorescence images stained for pNF-H, MAP-2 and DAPI; scale bars, 100 μm. ***, P<0.001. Error bars indicate standard error.

### DETAILED DESCRIPTION

The disclosure provides therapeutics and methods of using such therapeutics to treat, prevent, or ameliorate a symptom associated with, any of a variety of tumor-forming cancers that plague humans, other mammals and vertebrates, and all forms of animal life. The therapeutics are modulators (e.g., inhibitors) of β adrenergic receptors and/or muscarinic receptors involved in sympathetic and parasympathetic innervations of tumor tissue. More particularly with respect to inhibitors of β adrenergic receptors, the target(s) of inhibition is a β₂-adrenergic receptor and/or a β₃-adrenergic receptor. Without wishing to be bound by theory and speaking in general terms about typical effects, inhibition of β adrenergic receptor function inhibits tumor initiation and inhibition of muscarinic receptor activity inhibits tumor cell metastasis. Before providing a detailed description of the subject matter of the claims and the data provided in the working examples, key definitions of terms used throughout the disclosure are provided.

### I. DEFINITIONS

“Tumor”, as used herein, refers to any neoplastic cell growth or proliferation, whether malignant or benign, and to all pre-cancerous and cancerous cells and tissues.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, liver cancer, vulval cancer, thyroid cancer, esophageal cancer, hepatic carcinoma and various types of head and neck cancer.

As used herein, the phrase “metastatic cancer” is defined as a cancer that has the potential to, or has begun to, spread to other areas of the body. A variety of cancers can metastasize, but the most common metastasizing cancers are breast, lung, renal, multiple myeloma, thyroid and prostate. By way of example, other cancers that have the potential to metastasize include, but are not limited to, adenocarcinoma; blood cell malignancies, including leukemia and lymphoma; head and neck cancers; gastrointestinal cancers, including esophageal cancer, stomach cancer, colon cancer, intestinal cancer, colorectal cancer, rectal cancer, pancreatic cancer, liver cancer, cancer of the bile duct or gall bladder; malignancies of the female genital tract, including ovarian carcinoma, uterine endometrial cancers, vaginal cancer, and cervical cancer; bladder cancer; brain cancer, including neuroblastoma and glioma; sarcoma, osteosarcoma; and skin cancer, including malignant melanoma and squamous cell cancer.

The term “therapeutically effective amount” as used herein refers to an amount of a therapeutic agent to treat, ameliorate, or prevent a desired disease or condition, or to exhibit a detectable therapeutic or preventative effect. The effect is detected by, for example, a reduction in tumor size. The effect is also detected by, for example, chemical markers or antigen levels. Therapeutic effects also include reduction in physical symptoms, such as decreased body temperature. The precise effective amount for a subject will depend upon the subject's size and health, the nature and extent of the condition, the therapeutics or combination of therapeutics selected for administration, and other variables known to those of skill in the art. The effective amount for a given situation is determined by routine experimentation and is within the judgment of the clinician. For purposes of the present disclosure, an effective dose will generally be from about 0.01 mg/kg to about 5 mg/kg, or about 0.01 mg/kg to about 50 mg/kg or about 0.05 mg/kg to about 10 mg/kg of the compositions of the present disclosure in the individual to which it is administered.

The term “antibody” is used in the broadest sense of a peptide of, or derived in part from, an immunoglobulin that is capable of specifically binding to at least one ligand or binding partner. Exemplary antibodies include a fully assembled antibody (polyclonal or monoclonal), an antibody fragment that can bind antigen, e.g., Fab, Fab′, F(ab′)2, Fab′-SH, Fv, a single-chain antibody, a single-chain variable fragment (scFv), a linear antibody, a chimera, a humanized antibody, a human antibody, a peptibody, a diabody, and a recombinant polypeptide comprising any of the foregoing antibody forms.

The term “tumor initiation” refers to the process early in a primary or secondary tumor's development wherein cancer cells create an environment for their proliferation by recruiting stromal cell components required for survival and growth, including fibroblasts, immune cells, pericytes, endothelial cells, nerve cells, blood vessels, and inflammatory cells.

The term “perioperative” is defined as the time period describing the duration of a patient's surgical procedure. The perioperative period refers to the period during which care is given immediately before, during, and immediately after a surgical procedure (e.g., cancer surgery). The perioperative period begins at the time of admission to a healthcare facility (e.g., a hospital) for a surgical procedure, about 1 hour before admission, about 2 hours before admission, about 4 hours before admission, about 6 hours before admission, about 8 hours before admission, about 12 hours before admission, about 1 day before admission, about 2 days before admission, about 3 days before admission, about 4 days before admission, about 5 days before admission, about 6 days before admission, or about 1 week before admission. The perioperative period ends at the time of release from an immediate post-operative care facility (e.g., a recovery room), about 1 hour post-release, about 2 hours post-release, about 4 hours post-release, about 6 hours post-release, about 8 hours post-release, about 10 hours post-release, about 12 hours post-release, about 1 day post-release, about 2 days post-release, about 3 days post-release, about 4 days post-release, about 5 days post-release, about 6 days post-release, about 1 week post-release, about 2 weeks post-release, about 3 weeks post-release, or about 4 weeks post-release.

### II. ANTIBODY INHIBITORS

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized in homogeneous culture, uncontaminated by other immunoglobulins with different specificities and characteristics.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al., Nature, 256:495 [19751, or may be made by recombinant DNA methods (see, e.g., U.S. Pat. No. 4,816,567). The “monoclonal antibodies” may also be isolated from phage antibody libraries using the techniques described in Clackson et al., Nature, 352:624628[1991] and Marks et al., J. Mol. Biol., 222:581-597 (1991), for example.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (Zapata et al., Protein Eng., 8(10):1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two “Single-chain Fv” or “scFv” antibody fragments, each comprising a VH and VL domain of an antibody, wherein these domains are present in a single polypeptide chain. Preferably, the Fv polypeptide further comprises a polypeptide linker between the VH and VL domains that enables the Fv to form the desired structure for antigen binding. For a review of scFvs see Pluckthun in The Pharmacology of Monoclonal Antibodies, vol. 1 13, Rosenburg and Moore eds., Springer-Verlag, New York, pp. 269-315 (1994), incorporated herein by reference.

The term “diabodies” refers to small antibody fragments with two antigen-binding sites, which fragments each comprise a heavy-chain variable domain (VH) connected to a light-chain variable domain (VL) in the same polypeptide chain (VH VL). By using a linker that is too short to allow pairing between the two domains on the same chain, the domains are forced to pair with the complementary domains of another chain to create two antigen-binding sites in a molecule having two chains. Diabodies are described more fully in, for example, EP 404,097; WO 93/11161; and Hollinger et al., Proc. Natl. Acad. Sci. USA, 90:6444-6448 (1993).

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. Collectively, the six CDRs confer antigen-binding specificity to the antibody. Even a single variable domain (or half of an Fv comprising only three CDRs specific for an antigen), however, has the ability to recognize and bind antigen at lower affinity, and is contemplated as a form of receptor inhibitor disclosed herein.

In addition to the variable domains of a light and heavy chain, a Fab fragment contains the constant domain of the light chain and the first constant domain (CH1) of the heavy chain. Fab fragments differ from Fab′ fragments by the addition of a few residues at the carboxy terminus of the heavy chain CH 1 domain, including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

By “neutralizing antibody” is meant an antibody molecule that is able to eliminate or significantly reduce an effector function of a target antigen to which it binds. Accordingly, a “neutralizing” anti-target antibody is capable of eliminating or significantly reducing a target effector function, such as enzyme activity, ligand binding, or intracellular signaling.

As provided herein, the compositions for, and methods of, treating cancer metastasis and/or cancer engraftment may utilize one or more antibodies used singularly or in combination with other therapeutics to achieve the desired effects. Antibodies according to the present disclosure may be isolated from an animal producing the antibody as a result of either direct contact with an environmental antigen or immunization with the antigen. Alternatively, antibodies may be produced by recombinant DNA methodology using one of the antibody expression systems well known in the art (See, e.g., Harlow and Lane, Antibodies: A Laboratory Manual, Cold Spring Harbor Laboratory (1988)). Such antibodies may include recombinant IgGs, chimeric fusion proteins having immunoglobulin derived sequences or “humanized” antibodies that may all be used for the treatment of cancer metastasis and/or cancer engraftment according to the disclosure. In addition to intact, full-length molecules, the term “antibody” also refers to fragments thereof (such as scFv, Fv, Fd, Fab, Fab′ and F(ab)′2 fragments) or multimers or aggregates of intact molecules and/or fragments that bind to any of a β₂ adrenergic receptor, a β3 adrenergic receptor, or an M1 muscarinic receptor. These antibody fragments bind antigen and may be derivatized to exhibit structural features that improve their bioavailability by facilitating clearance and uptake, e.g., by incorporation of galactose residues.

In other embodiments of the present disclosure, humanized anti-β₂ adrenergic receptor, anti-β₃ adrenergic receptor, and anti-M1 receptor monoclonal antibodies are provided. The phrase “humanized antibody” refers to an antibody derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

The antigen to be used for production of antibodies may be an intact β₂ adrenergic receptor, β₃ adrenergic receptor, M1 receptor, or a fragment thereof that retains the desired epitope, optionally fused to another polypeptide that allows the epitope to be displayed in its native conformation.

A. Polyclonal Antibodies

Polyclonal antibodies are preferably raised in animals by multiple subcutaneous (sc) or intraperitoneal (ip) injections of the relevant antigen and an adjuvant. An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor, using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg or 5 μg of the protein or conjugate (for a rabbit or a mouse, respectively) with 3 volumes of Freund's complete adjuvant and injecting the solution intradermally at multiple sites. One month later, the animal is boosted with ⅕ to 1/10 the original amount of peptide or conjugate in Freund's complete adjuvant by subcutaneous injection at multiple sites. At 7-14 days post-booster injection, the animal is bled and the serum is assayed for antibody titer. An animal is boosted until the titer plateaus. Preferably, the animal is boosted with the same conjugate, but conjugation of the peptide bearing the antigenic site to a different immunogenic protein and/or through a different cross-linking reagent is contemplated. A conjugate also can be made in recombinant cell culture as a protein fusion. Also, an aggregating agent such as alum is suitably used to enhance the immune response.

B. Monoclonal Antibodies

Monoclonal antibodies may be made using the hybridoma method first described by Kohler et al., Nature, 256:495 (1975), or may be made by recombinant DNA methods.

In the hybridoma method, a mouse or other appropriate host animal, such as a hamster or macaque monkey, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), but lymphocytes are HGPRT⁺ (or HPRT⁺), the culture medium for the hybridomas typically will include hypoxanthine, aminopterin, and thymidine (HAT medium), to prevent the growth of the parental HGPRT-deficient myeloma cells. Continued growth of the cells distinguishes immortal hybridomas from the lymphocytes.

A preferred myeloma cell is that which fuses efficiently, supports stable high-level production of antibody by the selected antibody-producing cells, and remains sensitive to a selective medium. Human myeloma and mouse-human heteromyeloma cell lines also have been described for the production of human monoclonal antibodies (Brodeur et al., Monoclonal Antibody Production Techniques and Applications, pp. 51-63 (Marcel Dekker, Inc., New York, 1987)).

Medium from cultures of typically single hybridoma cells (obtained, e.g., by limiting dilution) is assayed for production of monoclonal antibodies directed against the antigen. Preferably, the binding specificity of a monoclonal antibody produced by a hybridoma cell is determined by immunoprecipitation or by an in vitro binding assay, such as radioimmunoassay (RIA) or enzyme-linked immunoabsorbent assay (ELISA). The binding affinity of the monoclonal antibody can, for example, be determined by Scatchard analysis (Munson et al., Anal. Biochem., 107:220 (1980)).

After hybridoma cells are identified that produce antibodies of the desired specificity, affinity, and/or activity, the clones may be subcloned by limiting dilution procedures (if not already a homogeneous cell population) and grown by standard methods (Goding, Monoclonal Antibodies: Principles and Practice, pp. 59-103 (Academic Press, 1986)). Suitable culture media for this purpose include, for example, D-MEM and RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies may be isolated and sequenced from the hybridoma cells using conventional procedures (e.g., by using oligonucleotide probes that are capable of binding specifically to genes encoding the heavy and light chains of the monoclonal antibodies). Once isolated, the DNA may be placed into expression vectors, which are then transfected into host cells such as Escherichia coli cells, simian COS cells, Chinese hamster ovary (CHO) cells, or myeloma cells that preferably do not otherwise produce immunoglobulin protein, to obtain the synthesis of monoclonal antibodies in the recombinant host cells. Recombinant production of antibodies is well known in the art.

Amino acid sequence variants of the desired antibody may be prepared by introducing appropriate nucleotide changes into the encoding DNA, or by peptide synthesis. Such variants include, for example, deletions from, and/or insertions into and/or substitutions of, residues within the amino acid sequences of the antibodies. Any combination of deletion, insertion, and substitution is made to arrive at the final construct, provided that the final construct possesses the desired characteristics, including binding specificity for a β2 or β3 adrenergic receptor or to a muscarinic receptor. The amino acid changes also may alter post-translational processing of the humanized or variant antibody, such as changing the number or position of glycosylation sites. Typical variants will have no more than 10, no more than 5, no more than 2, or no more than 1 residue that fails to correspond to the sequence of the non-variant cognate antibody.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier-prepared variant or a non-variant version of the antibody.

Because chimeric or humanized antibodies are less immunogenic in humans than the parental mouse monoclonal antibodies, they can be used for the treatment of humans with far less risk of anaphylaxis. Thus, these antibodies may be preferred in therapeutic applications that involve in vivo administration to a human.

Chimeric monoclonal antibodies, in which the variable immunoglobulin (Ig) domains of a mouse monoclonal antibody are fused to human constant Ig domains, can be generated using standard procedures known in the art (see Morrison, S. L., et al., Chimeric Human Antibody Molecules; Mouse Antigen Binding Domains with Human Constant Region Domains, Proc. Natl. Acad. Sci. USA 81, 6841-6855 (1984); and, Boulianne, G. L., et al, Nature 312, 643-646 (1984)). Although some chimeric monoclonal antibodies have proved less immunogenic in humans, the mouse variable Ig domains can still lead to a significant human anti-mouse response.

### III. INVOLVEMENT OF THE AUTONOMIC NERVOUS SYSTEM IN TUMOR INITIATION AND PROGRESSION TOWARD METASTASIS

Adrenergic activity delivered by the sympathetic nervous system (SNS) is required for HSC egress from the bone marrow under homeostasis or when enforced by granulocyte-colony stimulating factor (G-CSF), a cytokine used clinically to mobilize HSCs in blood for stem cell transplantation in caring for cancer patients. G-CSF has also been reported to mobilize tumor cells from the bone marrow. Based on observed parallels between the behaviors of healthy stem cells and cancerous tumor-initiating cells, autonomic neural signals were examined for an influence on the development and spreading of the primary tumor. Cancer cells seize and re-shape the healthy tissue microenvironment to promote their growth (inappropriate growth, from the perspective of an organism), invasion and ultimate metastasis. While multiple stromal contributions to cancer progression have been examined, the role of nerves in the developing tumor remains unclear. Disclosed herein are data establishing new autonomic nerve fiber formation in the primary tumor as a pivotal event that regulates cancer (e.g., prostate cancer) initiation and its dissemination. Using chemical or surgical prostate sympathectomy, adrenergic sympathetic neo-fiber development has been shown to be required for tumor initiation in xenogeneic orthotopic and transgenic mouse models. Moreover, tumor cell engraftment was impaired in hosts lacking the β2- and β3-adrenergic receptors. Tumors were also invaded by parasympathetic cholinergic fibers which, by contrast, regulated and/or promoted tumor cell motility or migration, lymph node invasion and metastasis to distant organs. Cholinergic-induced prostate cancer-spreading, i.e., metastasis, was dramatically inhibited by pharmacologic blockade or genetic disruption of the type 1 muscarinic receptor in the stroma. Stated in the alternative, metastatic tumors and overall survival were dramatically improved by the pharmacologic blockade or genetic disruption of the stromal type-1 muscarinic receptor. These results show that intratumor nerves from the two autonomic branches exert distinct, important functions in the tumor microenvironment, and offer therapeutic avenues to control prostate cancer development.

It is known in the art that perineural invasion confers a poor prognosis in prostate cancer and other carcinomas by providing a gateway toward hematogenous spread. The present results, in contrast, indicate the reverse, i.e., that the prostate tumor itself is invaded by nerves which, in turn, regulate tumor cell survival, proliferation and metastases to distant sites.

The stimulation of tumor nerve formation by G-CSF, a hematopoietic growth factor often used to support hematopoietic recovery in patients that have received cancer chemotherapy, was unexpected but consistent with the reported expression of its receptor on neurons where it promotes neuronal survival (not formation) during ischemia or pain sensation via sensory fibers surrounding sarcoma tumor in the skin. The disclosure shows that G-CSF can also act as a growth factor for autonomic peripheral nerves in the tumor microenvironment.

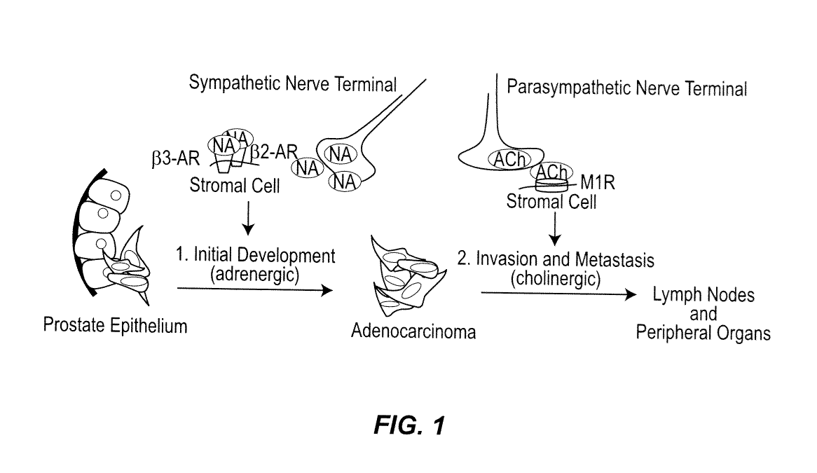

Distinct functions for the two branches of the autonomic nervous system in tumor initiation and progression toward metastasis (FIG. 1) have been identified herein. Disclosed herein are the first results useful in longitudinally evaluating the early events leading to metastasis from an experimental orthotopic tumor model. Whether prostate tumors were generated in a xenogeneic model, or arose spontaneously either in oncogene-driven transgenic mice or in primary human cancer, the recruitment of a dense network of autonomic nerves is apparent. These data indicate dual, complementary functions for autonomic innervation in prostate tumors where adrenergic fibers from the SNS are required for regulating tumor initiation through stromal β2 and β3 adrenergic receptors, and cholinergic fibers of the peripheral nervous system (PNS) drive tumor cell invasion, migration and metastasis to distant organs through stromal M1R (Chrm1), a muscarinic receptor. The data support new cancer methodologies for the prevention or treatment of an often-fatal disease, particularly when metastasis is likely or has occurred.

### IV. β ADRENERGIC RECEPTOR INHIBITORS

The term “β adrenergic receptor inhibitor” refers to any chemical, compound, or antibody that blocks the action of ligands binding to β adrenergic receptors, in particular β₁ adrenergic receptors, β₂ adrenergic receptors, and/or β₃ adrenergic receptors. In some embodiments, the administered β adrenergic receptor inhibitor significantly inhibits only a β₂ adrenergic receptor. In other embodiments, the administered β adrenergic receptor inhibitor significantly inhibits only a β₃ adrenergic receptor. In some embodiments, the administered β adrenergic receptor inhibitor significantly inhibits only a β₂ adrenergic receptor and a β₃ adrenergic receptor. In some embodiments, the administered β adrenergic receptor inhibitor significantly inhibits only a β₁ adrenergic receptor and a β₂ adrenergic receptor. In other embodiments, the administered β adrenergic receptor inhibitor significantly inhibits β₁, β₂, and β3 adrenergic receptors. In some embodiments, a β₂ adrenergic receptor-specific inhibitor is co-administered with a β₃ adrenergic receptor-specific inhibitor. In some embodiments, the β adrenergic receptor inhibitor is, optionally, butaxamine, propranolol, or ICI-118,551 (β₂ adrenergic receptor inhibitors), or SR 59230A (β₃ adrenergic receptor-selective inhibitor). In some embodiments, the β₂ adrenergic receptor inhibitor is an anti-β₂ adrenergic receptor antibody. In some embodiments, the β adrenergic receptor inhibitor is a nonspecific β adrenergic receptor inhibitor selected from the group consisting of alprenolol, bucindolol, carteolol, carvedilol, labetalol, nadolol, penbutolol, pindolol, sotalol, or timolol. In some embodiments, the β₃ adrenergic receptor inhibitor is an anti-β₃ adrenergic receptor antibody.

In some embodiments, the β adrenergic receptor inhibitor is a peptide. Peptides with β adrenergic receptor inhibiting activity can readily be screened from random synthetic peptide libraries by persons of ordinary skill in the art. The peptide can be about 5, about 10, about 15, about 20, about 25, about 30, about 35, about 40, about 45, about 50, about 55, about 60, about 70, or about 75 amino acids in length.

A. Inhibition of Tumor Initiation

In some embodiments, a β adrenergic receptor inhibitor is administered in an amount effective to inhibit tumor initiation. “Tumor initiation” refers to the process early in a primary or secondary tumor's development wherein cancer cells create an environment for their proliferation by recruiting stromal cell components required for survival and growth, including fibroblasts, immune cells, pericytes, endothelial cells, nerve cells, blood vessels, and inflammatory cells. Tumor initiation refers both to tumors coming into existence due to spontaneously arising cancer cells that develop into primary tumors and to secondary tumors coming into existence due to metastatic cancer cells that develop into secondary tumors. In some embodiments, a β₂ and/or a β₃ adrenergic receptor inhibitor is administered in an amount effective to inhibit recruitment of stromal cell components, including nerve cells, thereby inhibiting tumor initiation. In some embodiments, administration of a β₂ and/or a β₃ adrenergic receptor inhibitor prevents a spontaneously arising cancer cell from establishing a primary tumor. In some embodiments, administration of a β₂ and/or a β₃ adrenergic receptor inhibitor prevents a metastatic cancer cell from establishing a secondary tumor.

### V. MUSCARINIC RECEPTOR INHIBITORS

The term “muscarinic receptor inhibitor” refers to any chemical, compound, or antibody that blocks the action of at least one ligand binding to a muscarinic receptor, in particular an M1 muscarinic receptor. In some embodiments, the muscarinic receptor inhibitor is scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide, or telenzepine. In some embodiments, the muscarinic receptor inhibitor is an anti-M1 receptor antibody.

In some embodiments, the muscarinic receptor inhibitor is a peptide. Peptides with muscarinic receptor inhibiting activity can readily be screened from random synthetic peptide libraries by persons of ordinary skill in the art. The peptide can be about 5, about 10, about 15, about 20, about 25, about 30, about 35, about 40, about 45, about 50, about 55, about 60, about 70, or about 75 amino acids in length.

A. Inhibition of Metastasis

A muscarinic receptor inhibitor is suitable for administration to a cancer patient in an amount effective to inhibit metastasis. The tumor metastasis inhibitors of the present disclosure are administered to arrest further metastasis or are prophylactically administered in the case of an early cancer which has not yet metastasized. Muscarinic receptor inhibitors are also administered to prevent metastases following surgery or radiation therapy or to arrest micrometastases. The tumor metastasis inhibitors of the present disclosure may be applied to any type of malignant tumor. In various embodiments, metastasis of prostate cancer, breast cancer, melanoma, gastric cancer, colon cancer, or any other form of cancer capable of metastasis, is inhibited by administering a muscarinic receptor inhibitor.

### VI. ADMINISTRATION AND PREPARATION OF PHARMACEUTICAL FORMULATIONS

A. β Adrenergic Receptor Inhibitor/Muscarinic Receptor Inhibitor Combination Formulations

In some embodiments, pharmaceutical formulations comprising combinations of a β adrenergic receptor inhibitor and a muscarinic receptor inhibitor are provided. Combinations contemplated include M1 muscarinic receptor inhibitor/β₂ adrenergic receptor inhibitor, M1 muscarinic receptor inhibitor/β₃ adrenergic receptor inhibitor, M1 muscarinic receptor inhibitor/β₂ adrenergic receptor inhibitor/β₃ adrenergic receptor inhibitor, and M1 muscarinic receptor/non-specific adrenergic receptor inhibitor. In various embodiments, the M1 muscarinic receptor inhibitor is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide, and, telenzepine. In various embodiments, the β adrenergic receptor inhibitor is selected from the group consisting of an anti-β2 antibody, an anti-β3 antibody, butoxamine, propranolol, SR 59230A, alprenolol, bucindolol, carteolol, carvedilol, labetalol, nadolol, penbutolol, pindolol, sotalol, timolol and ICI-118,551.

B. Buffers, Additives, Excipients, and Stabilizers

The β adrenergic receptor inhibitors and/or muscarinic receptor inhibitors used in the practice of a method of the disclosure may be formulated into pharmaceutical compositions comprising a carrier suitable for the desired delivery method. Suitable carriers include any material which, when combined with a β adrenergic receptor inhibitor and/or muscarinic receptor inhibitor, retains the receptor inhibition activity and is nonreactive with the subject's immune system. Examples include, but are not limited to, any of a number of standard pharmaceutical carriers such as sterile phosphate-buffered saline solutions, bacteriostatic water, and the like. A variety of aqueous carriers may be used, e.g., water, buffered water, physiological saline, 0.4% saline, 0.3% glycine and the like, and may include other proteins for enhanced stability, such as albumin, lipoprotein, globulin, and the like, subjected to mild chemical modifications.

Exemplary inhibitor concentrations in the formulation may range from about 0.1 mg/ml to about 180 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the inhibitor may be prepared in a pH-buffered solution, for example, at pH ranging from about 4.5 to about 8.0, or from about 4.8 to about 6.5, or from about 4.8 to about 5.5, or alternatively about 5.0. Examples of buffers that are suitable within this pH range include acetate (e.g., sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

When the inhibitor is an antibody, a tonicity agent, which may also stabilize the antibody, may be included in the formulation. Exemplary tonicity agents include polyols, such as mannitol, sucrose or trehalose. Preferably, the aqueous formulation is isotonic, although hypertonic or hypotonic solutions are contemplated. Exemplary concentrations of the polyol in the formulation may range from about 1% to about 15% w/v.

When the inhibitor is an antibody, a surfactant may also be added to the antibody formulation to reduce aggregation of the formulated antibody and/or to minimize the formation of particulates in the formulation and/or to reduce adsorption. Of course, surfactants may also be used in formulations with non-antibody inhibitors. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e., antibody, buffer, polyol and surfactant) and is essentially free of one or more preservatives, such as benzyl alcohol, phenol, m-cresol, chlorobutanol and benzethonium Cl. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation, including specific binding to a β2 or β3 adrenergic receptor or to a muscarinic receptor. Acceptable carriers, excipients or stabilizers are nontoxic to recipients at the dosages and concentrations employed and include additional buffering agents, co-solvents, anti-oxidants including ascorbic acid and methionine, chelating agents such as EDTA, metal complexes (e.g., Zn-protein complexes), biodegradable polymers such as polyesters, and/or salt-forming counterions such as sodium.

Therapeutic formulations of the β adrenergic receptor inhibitors and muscarinic receptor inhibitors are prepared for storage by mixing the inhibitor having the desired degree of purity with physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration are preferably sterile. In some embodiments, the compositions of the disclosure may be sterilized by conventional, well-known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

C. Formulations with Additional Active Ingredients

The formulations herein may also contain more than one active compound as necessary for the particular indication being treated, preferably those with complementary activities that do not adversely affect each other. Such molecules are suitably present in combination in amounts that are effective for the purpose intended.

In some embodiments, a chemotherapeutic agent is co-administered or co-formulated with the β adrenergic receptor inhibitor and/or muscarinic receptor inhibitor wherein the anti-cancer agent is selected from the group consisting of Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon α-2a; interferon α-2b; interferon α-n1; interferon α-n3; interferon β-I a; interferon γ-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other chemotherapeutic agents include, but are not limited to, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; anagrelide; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; zanoterone; zilascorb; and zinostatin stimalamer. In particular embodiments, such anti-cancer agents are administered in combination with a β adrenergic receptor inhibitor and/or a muscarinic receptor inhibitor.

D. Sustained-Release Formulations

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The active ingredients may also be entrapped in a microcapsule prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

E. Lyophilized Formulations

When the inhibitor is an antibody, the process of freeze-drying is often employed to stabilize polypeptides for long-term storage, particularly when the polypeptide is relatively unstable in liquid compositions. A lyophilization cycle is usually composed of three steps: freezing, primary drying, and secondary drying; Williams and Polli, Journal of Parenteral Science and Technology, Volume 38, Number 2, pages 48-59 (1984). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter, the cake can be reconstituted prior to use.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration; Chen, Drug Development and Industrial Pharmacy, Volume 18, Numbers 11 and 12, pages 1311-1354 (1992).

For injection, the pharmaceutical formulation and/or medicament may be a powder suitable for reconstitution with an appropriate solution as described above. Examples of these include, but are not limited to, freeze-dried, rotary-dried or spray-dried powders, amorphous powders, granules, precipitates, or particulates. For injection, the formulations may optionally contain stabilizers, pH modifiers, surfactants, bioavailability modifiers and combinations of these.

F. Dosages

Therapeutically effective amounts of a composition will vary and depend on the severity of the disease and the weight, age, sex, diet, medical history, and general state (e.g., health) of the subject being treated, but generally range from about 1.0 μg/kg to about 100 mg/kg body weight, or about 10 μg/kg to about 30 mg/kg, or about 0.1 mg/kg to about 10 mg/kg or about 1 mg/kg to about 10 mg/kg per application. Administration can be daily, on alternating days, weekly, twice a month, monthly or more or less frequently, as necessary, depending on the response to the disorder or condition and the subject's tolerance of the therapy. Maintenance dosages over a longer period of time, such as 4, 5, 6, 7, 8, 10 or 12 weeks or longer may be needed until a desired suppression of disorder symptoms occurs, and dosages may be adjusted as necessary. The progress of this therapy is easily monitored by conventional techniques and assays, and is within the skill in the art.

Specific dosages may be adjusted depending on conditions of disease, the age, body weight, general health conditions, sex, diet, and medical history of the subject, dose intervals, administration routes, excretion rate, and combinations of drugs. Any of the above dosage forms containing effective amounts are well within the bounds of routine experimentation and, therefore, are within the scope of the instant disclosure.

G. Routes of Administration

The β adrenergic receptor inhibitors and/or muscarinic receptor inhibitors are administered by any suitable means, either systemically or locally, including via parenteral, subcutaneous, intrapulmonary, intramuscular, oral, and intranasal, and, if desired for local treatment, intralesional administration. Parenteral routes include intravenous, intraarterial, epidural, and intrathecal administration. In addition, the inhibitor is suitably administered by pulse infusion, particularly with declining doses of the inhibitor. Preferably, the dosing is given by injections, most preferably by intravenous or subcutaneous injections, depending in part on whether the administration is brief or chronic. Other administration methods are contemplated, including topical, particularly transdermal, transmucosal, rectal, oral or local administration, e.g., through a catheter placed close to the desired site. In some embodiments, the receptor inhibitor is administered intravenously in a physiological solution at a dose ranging between 0.01 mg/kg to 100 mg/kg at a frequency ranging from daily to weekly to monthly (e.g., every day, every other day, every third day, or 2, 3, 4, 5, or 6 times per week), a dose ranging from 0.1 to 45 mg/kg, 0.1 to 15 mg/kg or 0.1 to 10 mg/kg at a frequency of 2 or 3 times per week, or up to 45 mg/kg once a month.

H. Perioperative Administration

In various embodiments, the β adrenergic receptor inhibitors and/or muscarinic receptor inhibitors of the disclosure are administered during a perioperative period. In various embodiments, the perioperative period begins at the time of admission to a healthcare facility (e.g., a hospital) for a surgical procedure (e.g., cancer surgery), about 1 hour before admission, about 2 hours before admission, about 4 hours before admission, about 6 hours before admission, about 8 hours before admission, about 12 hours before admission, about 1 day before admission, about 2 days before admission, about 3 days before admission, about 4 days before admission, about 5 days before admission, about 6 days before admission, or about 1 week before admission. In various embodiments, the perioperative period ends at the time of release from an immediate post-care facility (e.g., a recovery room) after a surgical procedure, about 1 hour post-release, about 2 hours post-release, about 4 hours post-release, about 6 hours post-release, about 8 hours post-release, about 10 hours post-release, about 12 hours post-release, about 1 day post-release, about 2 days post-release, about 3 days post-release, about 4 days post-release, about 5 days post-release, about 6 days post-release, about 1 week post-release, about 2 weeks post-release, about 3 weeks post-release, or about 4 weeks post-release.

### EXAMPLES

The following examples are provided for illustration and are not intended to limit the scope of the disclosure. Example 1 provides data establishing the mouse model of cancer using orthotopic tumor generation in nude mice. Example 2 establishes the extent of sympathetic and parasympathetic innervation of an orthotopic prostate tumor. Example 3 establishes the role of the sympathetic nervous system in tumor (prostate) engraftment. Example 4 establishes the role of the parasympathetic nervous system in tumor (prostate) metastasis. Example 5 demonstrates that tumor metastasis is blocked by chemical inhibitors of the M1 muscarinic receptor. Example 6 establishes that human cancers (prostate) exhibit sympathetic and parasympathetic innervation similar to that seen in the mouse orthotopic tumor model. Example 7 shows that effective cancer treatments are provided that comprise administration of a β adrenergic receptor inhibitor, a muscarinic receptor inhibitor, or inhibitors of both receptor types. Example 8 shows that cholinergic agonists do not affect vascular hemodynamics.

### Example 1

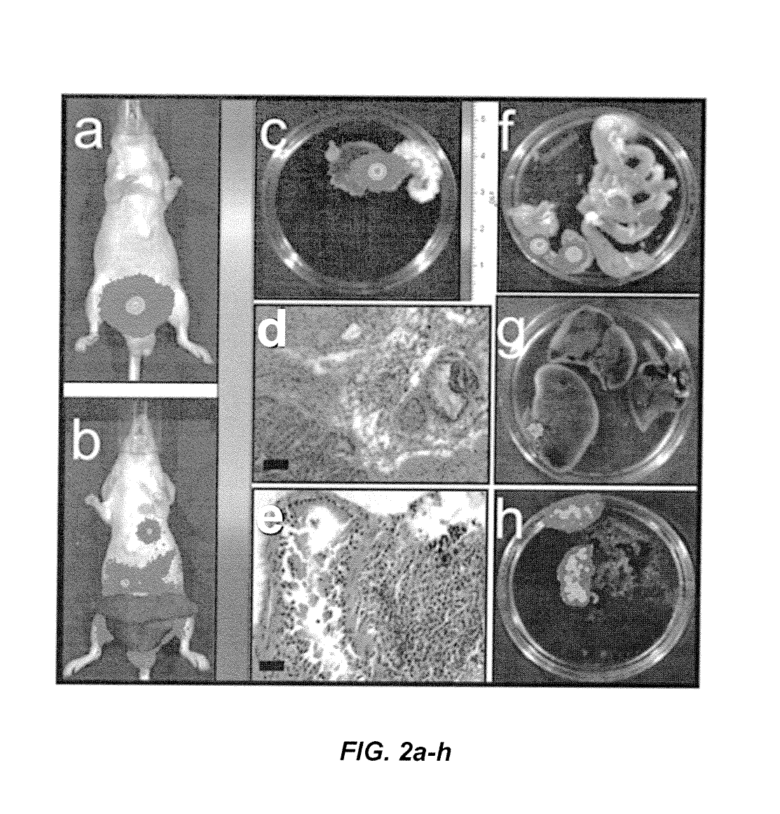

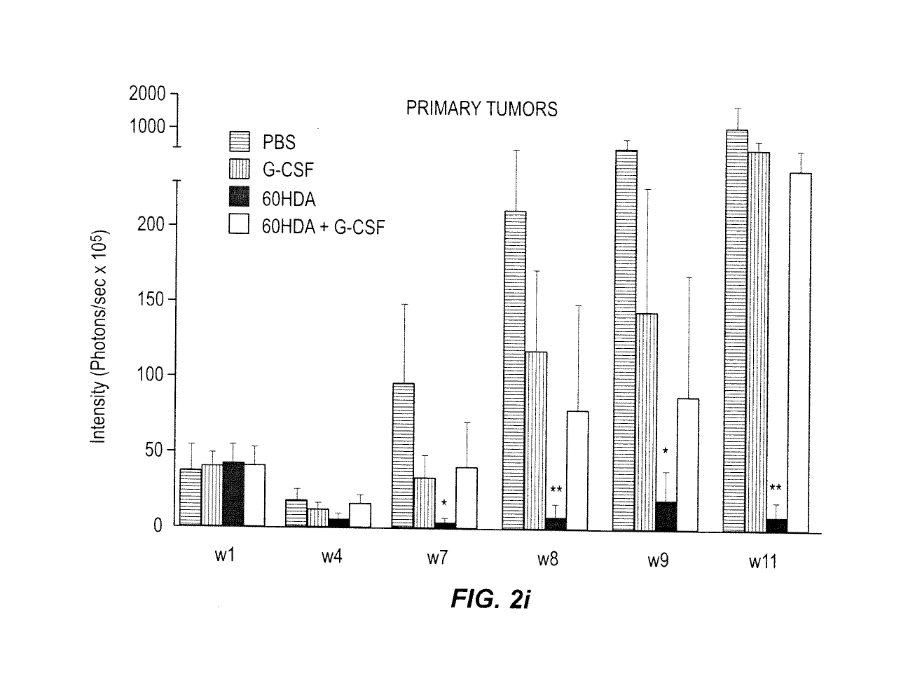

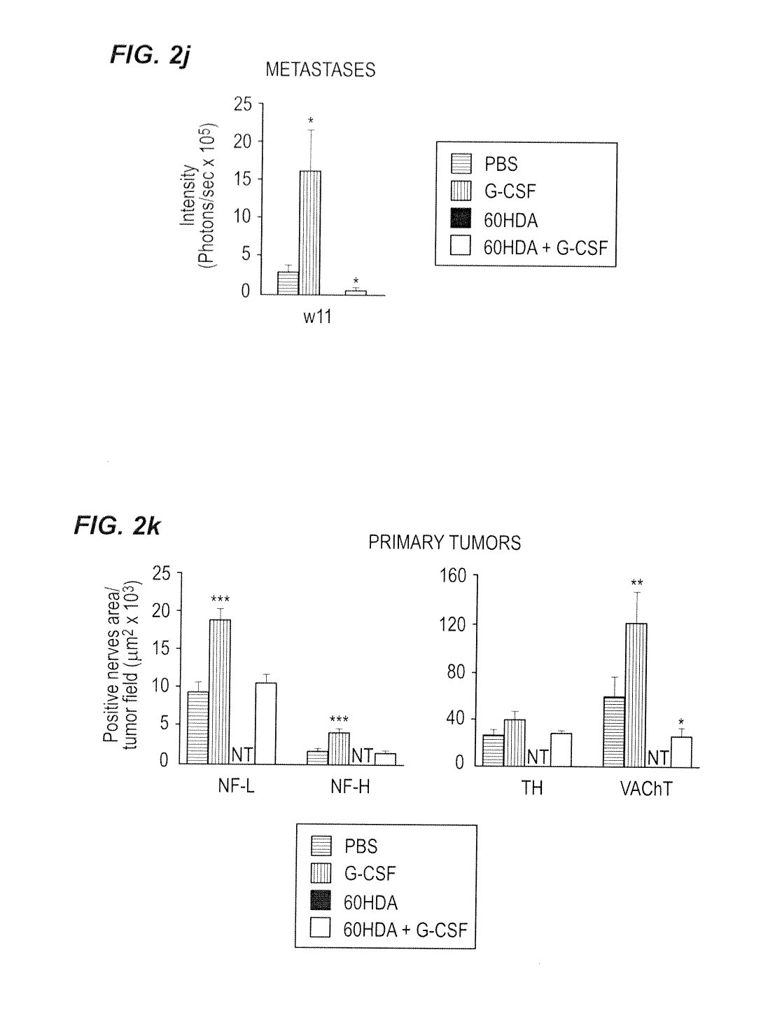

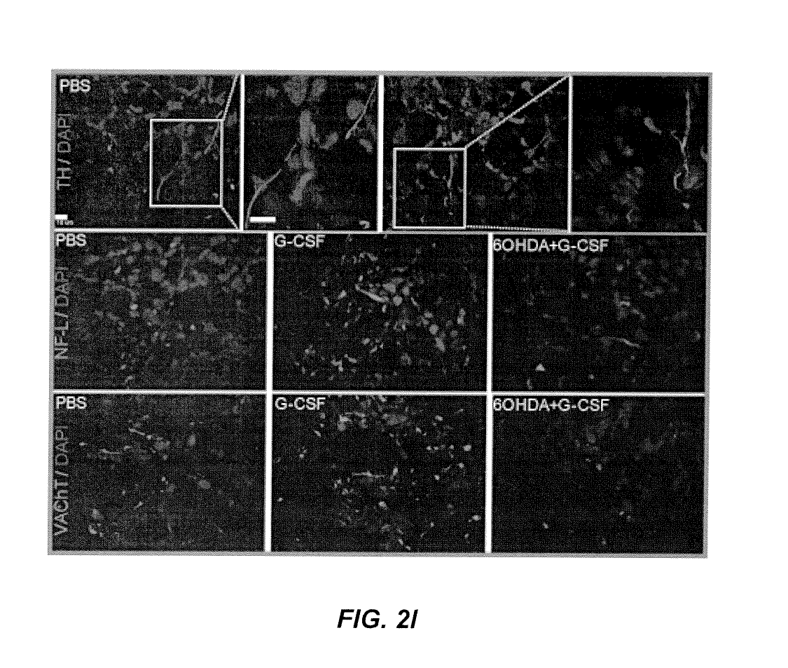

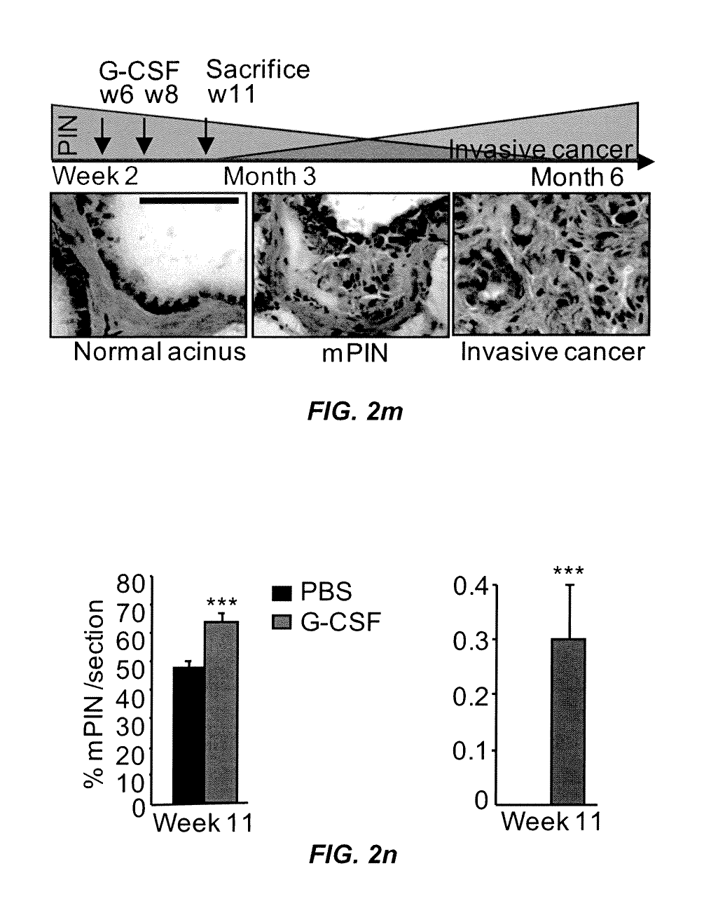

The microenvironment of a developing tumor is important in cancer development and metastasis. A xenogeneic orthotopic model of prostate cancer was established to provide a minimally disruptive microenvironment for this cancer in live mice. Implantation of PC-3 cells stably expressing luciferase (PC-3luc) in the prostate of immunodeficient nude (nu/nu) mice led to reproducible intracapsular prostate tumors that generated distant metastases to the intestine, liver, stomach, pancreas and lung at and beyond week 11 post-implantation (FIG. 2a-h).

### Mouse Strains

Balb/c nu/nu (Charles River laboratories) and cMyc (FVB-Tg(ARR2/Pbsn-MYC)7Key) mice (National Cancer Institute) were used in these studies. Immunodeficient B6.Cg-Foxn1^{nu+/-} heterozygous nude mice (Charles River Laboratories) were intercrossed with Adrb2^{tm1Bkk/J-/-} and Adrb3^{tm1Lowl/J-/-} to generate nu/nu mice lacking one or both adrenergic receptors, or with Chrm1^{tm1Stl-/-} obtained from the Jackson Laboratory. FVB-Tg(ARR2/Pbsn-MYC)7Key Chrm1^{tm1Stl-/-} and respective controls were also generated by intercrossing the two strains.

### Bioluminescence Imaging

In vivo and ex vivo bioluminescence imaging was performed and analyzed using an IVIS imaging system 200 series (Xenogen, Caliper Life Sciences, Hopkinton, Mass.). Bioluminescent signal was induced by i.p. injection of D-luciferin (150 mg/kg in PBS) 8 minutes prior to in vivo imaging. For ex vivo imaging, D-luciferin (300 mg/kg) was injected 7 minutes prior to necropsy. Organs of interest were immersed in a solution of D-luciferin at 150 mg/ml.

### Cell Culture

PC-3 cells stably transfected with the luciferase gene (PC-3luc) were grown in F12-Glutamax medium supplemented with 10% FBS, 1.5 g/l Bicarbonate sodium and 500 mg/ml G418 (Invitrogen, Carlsbad, Calif.). LNCaP cells expressing luciferase (Xenogen, Caliper Life Sciences, Hopkinton, Mass.) were grown according to manufacturer's recommendations in RPMI medium (ATCC#30-2001) supplemented with 10% FBS and 1% Penicillin/Streptomycin (Gibco, Carlsbad, Calif.). The promyelocytic cell line HL60 (ATCC #CCL-240™) and the histiocytic lymphoma U-937 (ATCC #CRL-1593.2™) were cultured according to manufacturer's recommendations.

### Histology and Immunofluorescence

Upon sacrifice, mouse prostate tissues were immersed in OCT medium (Tissue Teck®). Five-micrometer frozen sections were stained with hematoxylin-eosin (H&E). For immunofluorescence, unstained prostate sections were fixed with acetone or methanol. For staining of nerves, fixed sections were incubated in H₂O₂ to quench endogenous peroxidase and non-specific binding was blocked with goat serum in bovine serum albumin (BSA) solution and the avidin/biotin blocking kit (Vector Laboratories, Burlingame, Calif.). Sections were incubated with a rabbit anti-TH antibody (Millipore, Billerica, Mass.), or anti-VAChT antibody (Phoenix Pharmaceuticals, Inc., Burlingame, Calif.), or anti-NF-L antibody (Millipore), or anti-NF-H antibody (Abcam, Cambridge, Mass.) and then with a secondary biotinylated goat anti-rabbit antibody (Vector Laboratories). Signal was amplified using the Vectastain Elite ABC Kit (Vector Laboratories) and visualized using the Tyramide Signal Amplification kit for TRITC (PerkinElmer, Boston, Mass.).

For staining of vessels, fixed sections were blocked and amplified according to the same protocol described above. Mouse tissue sections were incubated with a rat monoclonal anti-CD34 antibody (Abcam) and then a goat anti-rat antibody (Vector Laboratories). Human sections were stained with a rabbit polyclonal anti-CD34 antibody (Abbiotec, San Diego, Calif.). For double staining of nerves and vessels, samples were incubated with allophycocyanin (APC) rat anti-mouse CD31 (BD Pharmingen, Grayson, Ga.) and then stained with antibodies against nerves as described above.

For proliferative cell quantification, sections were incubated with a rabbit polyclonal anti-Ki67 (Abcam) and then Alexafluor 568-conjugated goat anti-rabbit antibody (Molecular Probes, Carlsbad, Calif.).

For apoptotic cell quantification, prostate sections were fixed with 4% paraformaldehyde (PFA) and stained with the Mebstain Apoptosis Kit according to manufacturer's recommendations (MBL International, Woburn, Mass.). Basement membranes were stained with a rat polyclonal anti-α2laminin antibody (Abcam) followed by Alexafluor647-conjugated goat anti-rat antibody (Molecular Probes).

Brightfield images were captured and collected with a Zeiss Axioplan2 microscope (Carl Zeiss MicroImaging, Inc., Thornwood, N.Y.) and with a Q-imaging MP3.3 RTV color camera controlled by Zeiss AxioVision software. Full cMyc prostate sections were captured with a Zeiss Axioplan2IE and a Zeiss AxioCam MRc camera controlled by Zeiss AxioVision software. The system utilizes an encoded motorized stage that automates montage acquisition and stitching for high-resolution images of large areas.

Fluorescence images were captured and analyzed with a Yokogawa CSUX-A1 confocal scanner head equipped with four stack laser system and a piezoelectric focusing collar on a Zeiss work station. Images were collected with a Coolsnap HQ digital camera (Ropert Scientific, Munich, Germany). A Dell workstation with SlideBook software (Intelligent Imaging Innovations, Denver, Colo.) provided for synchronization of components, data acquisition and area quantification.

To evaluate neurite growth, cultures were fixed for 10 minutes at −20° C. with 5% glacial acetic acid/95% ethanol and then incubated with an anti-phosphorylated NF-H antibody (Sigma, clone NE14) and a rabbit anti-microtubule-associated protein-2 antibody (Chemicon), followed by Alexafluor488-conjugated goat anti-mouse antibody and Alexafluor568-conjugated goat anti-rabbit antibody (Molecular probes). For G-CSFR staining, mouse fetal neurons in culture were fixed with methanol and then incubated with a rabbit anti-G-CSFR (Santa-Cruz biotechnology, Santa Cruz, Calif.) and Alexafluor568-conjugated goat anti-rabbit antibody (Molecular probes). Neurite outgrowth was analyzed objectively from 40 random fields (field area=0.6 mm²) per growth condition using the “Neurite Outgrowth” module of Metamorph software (MDSAT).

### Proliferation Assay

A cell proliferation assay was performed on human PC-3 prostate tumor cells after 96 hours of treatment by G-CSF at three different concentrations by comparison with human leukemic cells (HL-60 and U937). Cell proliferation was quantified by a cell viability assay using MTT (3-[4,5-dimethylthiazole-2-yl]-2,5-diphenyltetrazolium bromide (Sigma). The percentage of viable cells was calculated by comparing the optical density (OD) of the treated samples with those incubated with vehicle. Methods were carried out as described by Magnon, et al., Cancer Res, 65, 4353-61 (2005); incorporated herein by reference in its entirety.

### RNA Extraction, RT-PCR and Q-PCR

Gene expression levels were analyzed from RNA extracted, using the TRIzol solution (Invitrogen, Carlsbad, Calif.), from PC-3, DU145, LNCaP cell lines or prostates from Balb/c nu/nu mice by quantitative real-time PCR, as described previously (Mendez-Ferrer, Nature, 452:442-447 (2008)). Primer sequences are provided in Table 1.

TABLE 1

| | | Sequence |
|---|---|---|
| | | Identifier |
| Gene | Primer Sequence | (SEQ ID NO) |
| hChrm1R | 5′-TGACCGCTACTTCTCCGTGACT | 1 |
| For | | |
| hChrm1R | 5′-CCAGAGCACAAAGGAAACCA | 2 |
| Rev | | |
| hChrm2R | 5′-TCACAAAACCTCTGACCTACCC | 3 |
| For | | |
| hChrm2R | 5′-TCCACAGTTCTCCACCCTACAA | 4 |
| Rev | | |
| hChrm3R | 5′-ACCATCCCTCAACTCCACCAAGT | 5 |
| For | | |
| hChrm3R | 5′-GGAAAACTGCCTCCATCGTC | 6 |
| Rev | | |
| hChrm4R | 5′-TCGCTATGAGACGGTGGAAA | 7 |
| For | | |
| hChrm4R | 5′-AGCACAACCAATAGCCCAAG | 8 |
| Rev | | |
| hChrm5R | 5′-GAAAGCAGCCCAGACACTGA | 9 |
| For | | |
| hChrm5 | 5′-AGCACAACCAACAGCCCAAG | 10 |
| Rev | | |
| hGCSFR | 5′-TCGGAAAGGTGAAGTAACTTGTCC | 11 |
| For | | |
| hGCSFR | 5′-TCCATGGGATCAAGACACAG | 12 |
| Rev | | |
| β-actin | 5′-TGTGATGGTGGGAATGGGTCAG | 13 |
| For | | |
| β-actin | 5′-TTTGATGTCACGCACGATTTCC | 14 |
| Rev | | |
| mChrm1R | 5′-CAGAAGTGGTGATCAAGATGCCTAT | 15 |
| For | | |
| mChrm1R | 5′-GAGCTTTTGGGAGGCTGCTT | 16 |
| Rev | | |
| mChrm2R | 5′-TGGAGCACAACAAGATCCAGAAT | 17 |
| For | | |
| mChrm2R | 5′-CCCCTGAACGCAGTTTTCA | 18 |
| Rev | | |
| mChrm3R | 5′-CCGCTCTACCTCTGTCCTTCA | 19 |
| For | | |
| mChrm3R | 5′-GGTGATCTGACTTCTGGTCTTGAG | 20 |
| Rev | | |
| mChrm4R | 5′-GTGACTGCCATCGAGATCGTAC | 21 |
| For | | |
| mChrm4R | 5′-CAAACTTTCGGGCCACATTG | 22 |
| Rev | | |
| mChrm5R | 5′-GGCCCAGAGAGAACGGAAC | 23 |
| For | | |
| mChrm5R | 5′-TTCCCGTTGTTGAGGTGCTT | 24 |
| Rev | | |
| Gapdh For | 5′-GCATGGCCTTCCGTGTTC | 25 |
| Gapdh Rev | 5′-CCTGCTTCACCACCTTCTTGA | 26 |

### Statistical Analyses

All values are reported as mean±sem. Statistical significance for three or more groups was assessed by a non-parametric one way ANOVA test (Kruskal-Wallis), followed by an unpaired Mann-Whitney test. Significance was set at p<0.05. The Kaplan-Meier method was used for survival curve analysis, and the log-rank (Mantel-Cox) test was used to determine the statistical significance of difference between survival curves using Graphpad Prism 5 software.

### Results

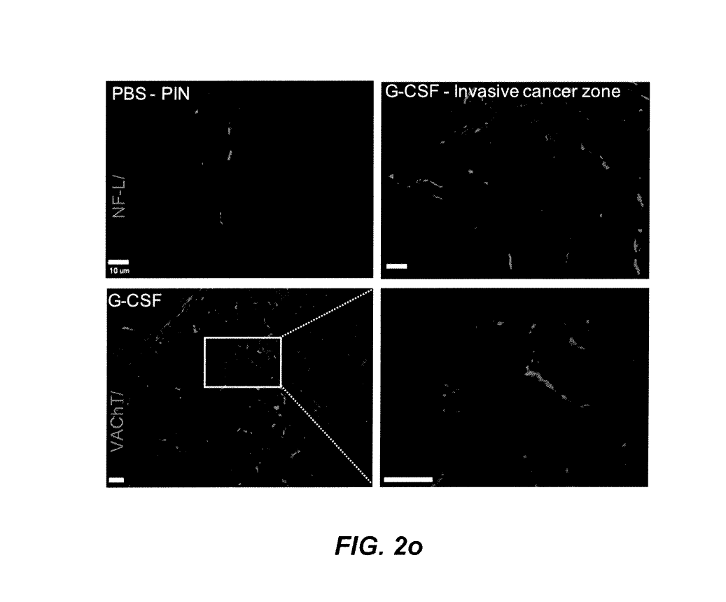

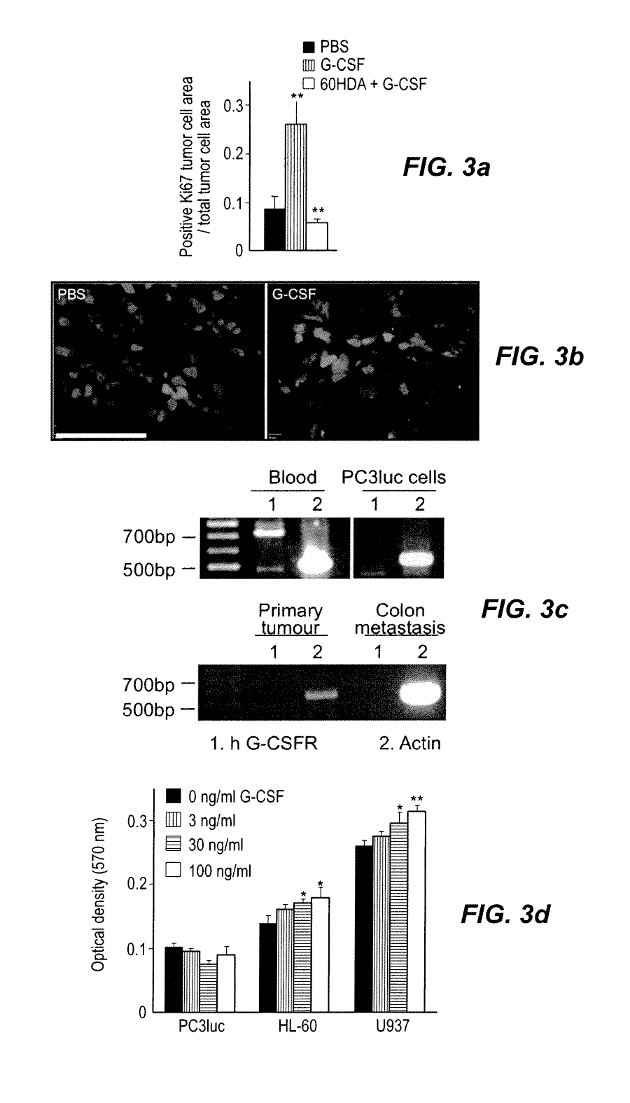

G-CSF was administered at a dose and regimen used to mobilize HSCs (250 μg/kg/day, for four consecutive days). The G-CSF significantly increased tumor cell proliferation in the primary tumors, as determined by Ki67 staining (FIG. 3a, b), and lymph node invasion and metastases were assessed by serial bioluminescence analyses (FIG. 2j). G-CSF-induced metastasis was not cell-autonomous because the G-CSF receptor (encoded by CSF3R) was not expressed in PC-3luc tumor cells and G-CSF could not stimulate the proliferation of PC-3luc tumor cells in vitro, as it did in vivo (FIG. 3c-d). Although G-CSF treatment increased tumor cell proliferation, it did not affect the size of the primary tumor, indicating that it influences tumor cell egress and metastatic efficiency from the stroma (FIG. 2i, FIG. 3a, b). Thus, G-CSF promotes prostate cancer cell proliferation and spreading by acting on the tumor microenvironment.

These results establish an orthotopic model for prostate cancer development that allows rapid and accurate assessment of the microenvironmental effects on cancer progression. Heterotopic mouse tumor models, e.g., induction of mouse hind-limb tumors, lack the proper microenvironmental context offered by the orthotopic model.

### Example 2

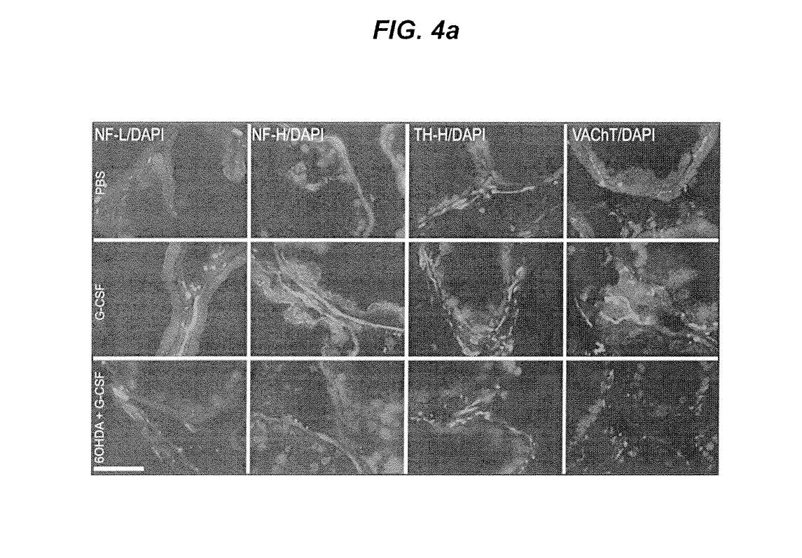

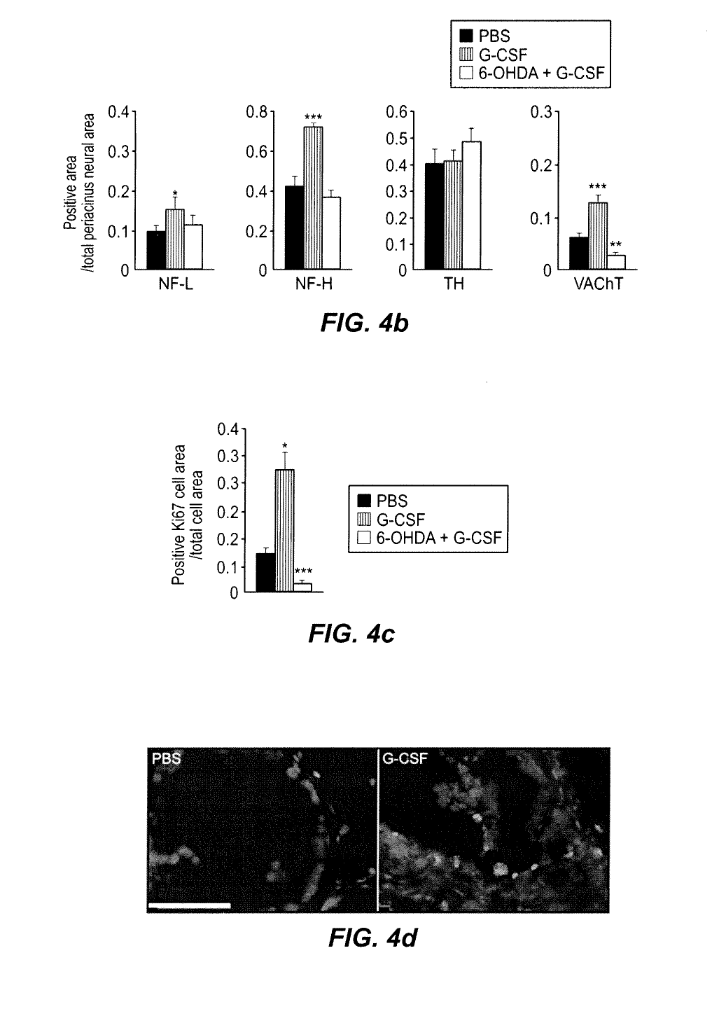

The healthy prostate stroma receives abundant innervation from the autonomic nervous system that can influence prostate weight. The presence and distribution of autonomic nerves in healthy and tumor-laden prostates was assessed. More particularly, sections of PC-3luc primary prostate tumors were examined for the presence of nerve fibers. Staining for tyrosine hydroxylase (TH) and for the vesicular acetylcholine transporter (VAChT) revealed abundant infiltration of sympathetic (adrenergic) and parasympathetic (cholinergic) fibers, respectively, in primary tumors (FIG. 2k, l). Specificity was confirmed by expression of the neuron-specific cytoskeletal subunits of neurofilament-L (NF-L), which identifies neo-fibers (FIG. 2k, l). By contrast, most autonomic neural fibers in tumor-free adult prostate tissues were positive for NF-H, a marker of more mature fibers (FIG. 4). Interestingly, prostate tumors from mice treated with G-CSF exhibited significantly higher densities of newly formed NF-L+ nerves (FIG. 2k, l). Thus, the disclosure provides a method of diagnosing cancer, e.g., prostate cancer, comprising detecting a neurofilament-L marker in a tissue subject to diagnostic assay. In some embodiments, the method further comprises identifying the tissue as exhibiting an absence of a marker for neurofilament H. Adrenergic varicose fibers stained for tyrosine hydroxylase (TH) activity were lined beneath the epithelium of healthy prostate acini within muscle bundles of the stroma (FIG. 4a). In contrast, cholinergic fibers expressing the vesicular acetylcholine transporter (VAChT) tightly surrounded epithelial cells, forming close bonds with smooth muscles and epithelial cells (FIG. 4a). These represented true nerve fibers since they also expressed the neuron-specific cytoskeletal subunits of neurofilament-L (NF-L) and neurofilament-H (NF-H) (FIG. 4a, b).

Remarkably, sections of primary prostate tumors revealed abundant infiltration of sympathetic (TH+) and parasympathetic (VAChT+) fibers inside the tumor, indicating robust tumoral neo-nerve formation (FIG. 2k, l). Most of the tumor neural fibers were positive for NF-L, which has been reported to identify neo-fibers, as compared to NF-H, a putative marker of mature fibers (FIG. 2k, l). Interestingly, prostate tumors from mice treated with G-CSF exhibited a significantly higher density of newly formed nerves, as determined by NF-L staining (FIG. 2k, l). In the healthy (tumor-free) prostate, a higher density of NF-L+ nerve fibers three weeks after G-CSF administration was observed, compared with PBS control (FIG. 4b), and this correlated with the proliferation of normal epithelial cells (Ki67+) (FIG. 4c, d).

### Fetal Neuronal Cultures

Neuronal cultures were prepared from embryonic day 15 fetal neocortex as described in Dobrenis et al., J Neurosci. Res. 82(3):306-315 (2005), incorporated herein by reference. Briefly, after enzymatic and mechanical tissue dissociation, cells were plated at 2.5×10⁴/cm² (day 0) and treated with 20 μg/ml 5-fluoro-2′ deoxyuridine and 50 μg/ml uridine at day 4 for 24 hours to eliminate glial growth. At day 1, cultures were maintained in serum-free medium and incubated with G-CSF at different concentrations (0, 500, 1000 ng/ml) until day 7.

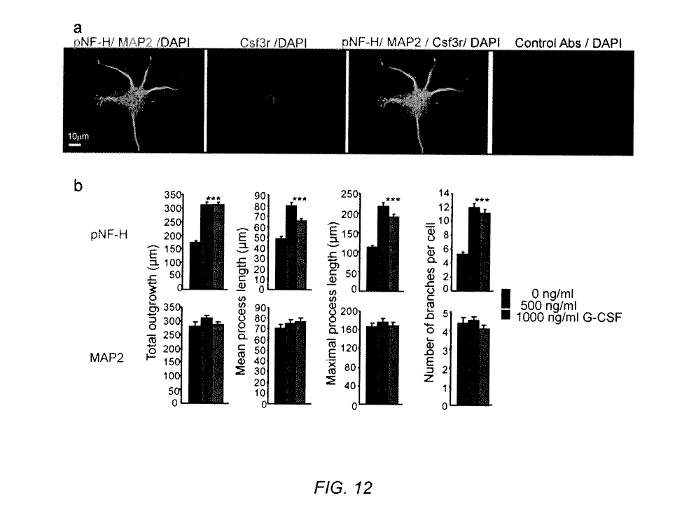

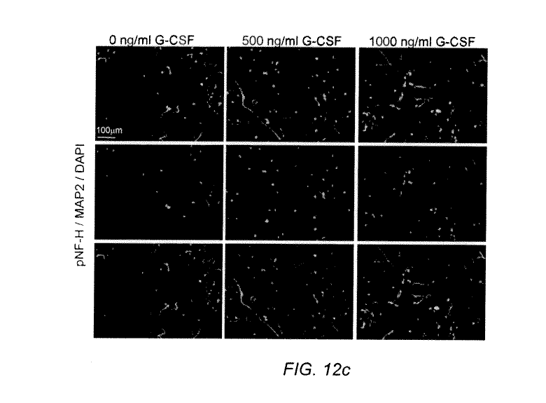

Because Csf3r is reportedly expressed on neurons, promoting survival during ischemia or pain by sensory fibers surrounding sarcoma tumor in the skin, the effect of Csf3r on neural outgrowth was examined. Cultures of fetal neurons that expressed Csf3r (FIG. 12a) were prepared. After cultivation for 6 days, non-biased, automated analyses revealed greater (about 2-fold) axonal outgrowth in G-CSF-treated neurons compared with vehicle-treated controls, whereas dendrite outgrowth was unchanged (FIG. 12b,c). These in vitro results, consistent with our in vivo observations in prostate tumors, indicate that G-CSF directly promotes axonal growth.

To investigate further the activity of G-CSF on tumor development using a genetic mouse model, transgenic mice expressing human cMyc under the ARR2/probasin promoter expressed specifically in the prostate (hereafter cMyc mice) were evaluated. This transgenic model leads to the complete penetrance of mouse prostatic intraepithelial neoplasia (mPIN) from postnatal week 2, progressing to invasive adenocarcinomas between 3 to 6 months of age (FIG. 2m). Whereas G-CSF treatment had a modest, but significant, effect on the frequency of mPIN at 11 weeks of age, the number of invasive cancer zones, which are normally absent at this stage, was dramatically increased (FIG. 2n). The development of invasive cancer was associated with a broad infiltration of NF-L+ neo-fibers by comparison with control animals (FIG. 2o). Thus, the increased tumor/normal epithelial cell proliferation following G-CSF administration is associated with (i.e., is correlated to) the development of new nerve fibers in the primary tumor. Accordingly, a method of treating cancer in a subject such as a human patient or non-human animal (e.g., non-human mammal) comprises administering a prophylactically or therapeutically effective amount of an inhibitor of NF-L+ neo-fibers. In some embodiments, the method comprises administering a prophylactically or therapeutically effective amount of an inhibitor of G-CSF.

### Example 3

The role of the sympathetic nervous system (SNS) in tumor cell release was investigated.

### Animal Procedures

Human tumors were induced by orthotopic surgical implantations of 1×10⁵ PC-3luc cells into six- to eight-week-old Balb/c nu/nu mice. Ten days after cell injection (day 0), the animals were randomized into the different groups and received the appropriate drugs, as indicated. 6OHDA or vehicle was injected at day 0 (100 mg/kg) and day 2 (250 mg/kg), and human recombinant G-CSF treatment was begun on day 4 (Amgen, 250 μg/kg/day, every 12 hours, 8 divided doses, i.p., in 5% dextrose). A second cycle was administered eight weeks after graft and animals were sacrificed three weeks after the last injection of G-CSF (i.e., 11 weeks after graft). In other experiments, 2×10⁵ PC-3luc cells were orthotopically injected into nu/nu Adrβ2^{tm1Bkk/J+/+} Adrβ3^{tm1Lowl/J+/+}, nu/nu Adrβ2^{tm1Bkk/J-/-}, nu/nu Adrβ3^{tm1Lowl/J-/-} and nu/nu Adrβ2^{tm1Bkk/J-/-} Adrβ3^{tm1Lowl/J-/-}. In selected experiments, 2×10⁶ LNCaP-luc cells were injected in nu/nu Adrβ2^{tm1Bkk/J+/+} Adrβ3^{tm1Lowl/J+/+} and nu/nu Adrβ2^{tm1Bkk/J-/-} Adrβ3^{tm1Lowl/J-/-} mice. For the transgenic model, one-, two- or five-month-old c-Myc mice were injected with 6OHDA or surgically sympathectomized according to protocols described above and sacrificed 30 days later.

### Results

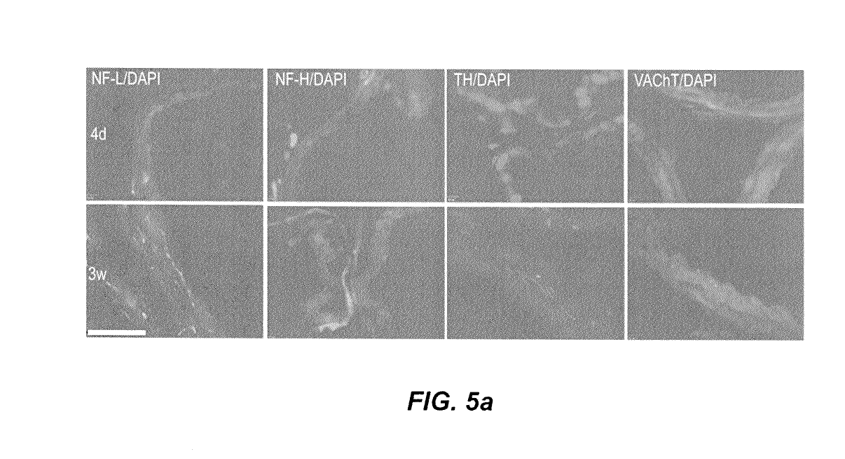

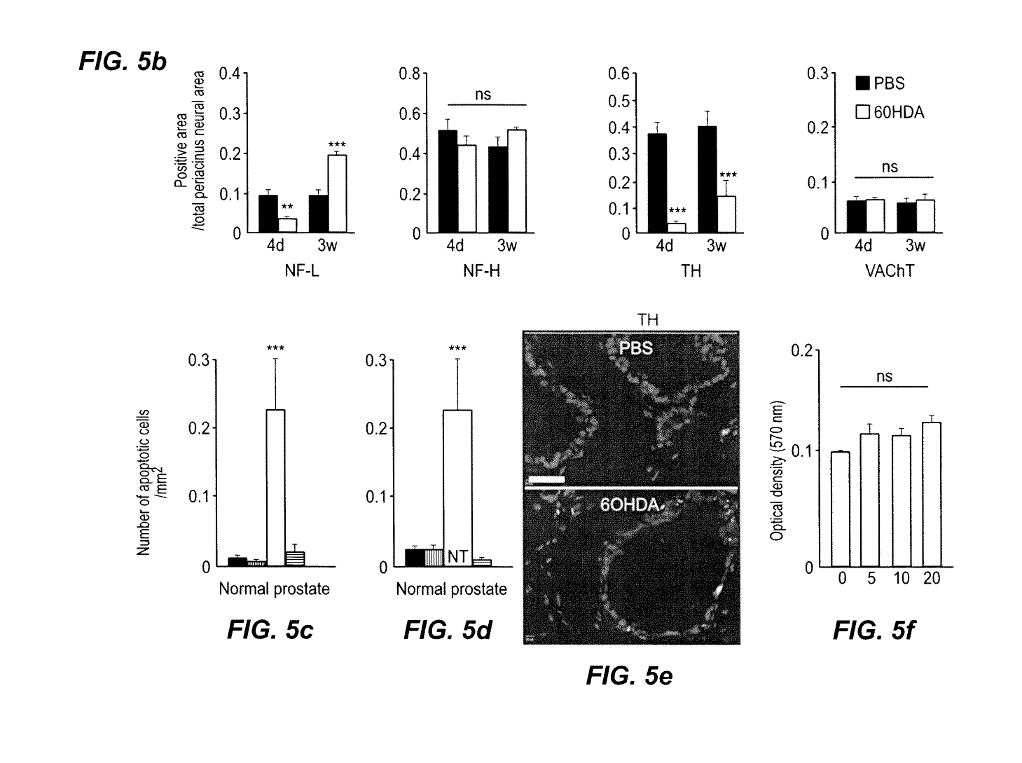

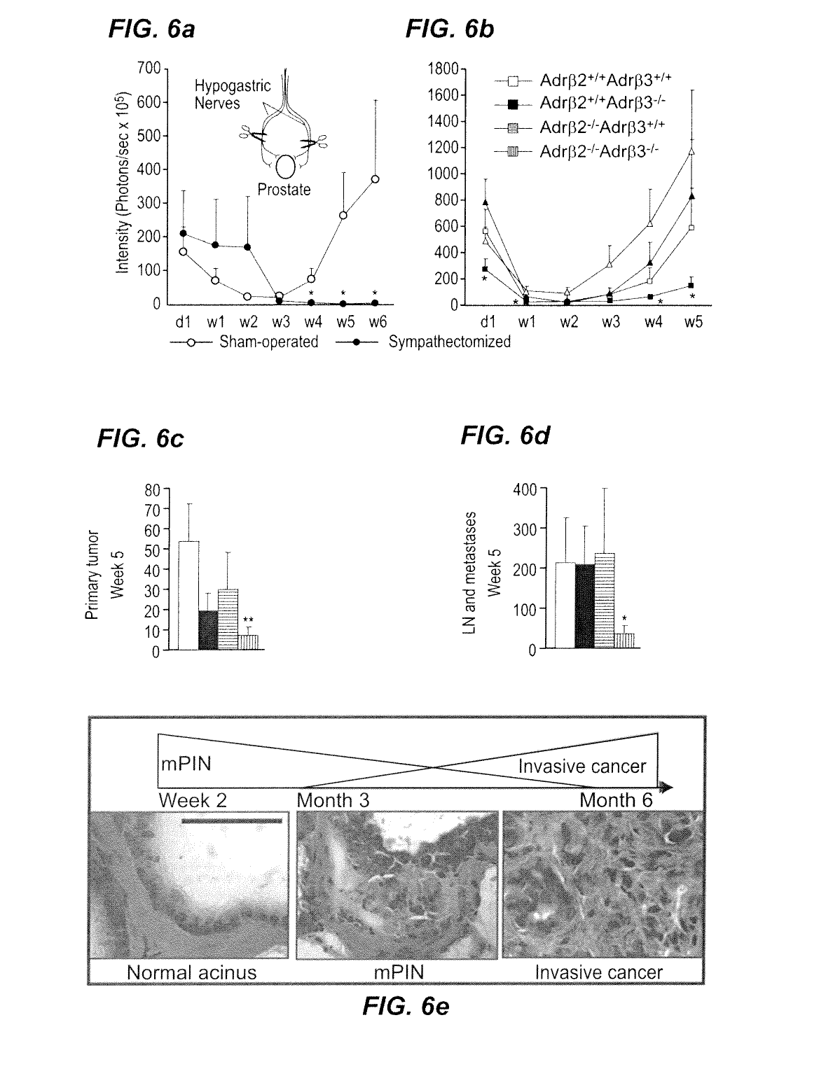

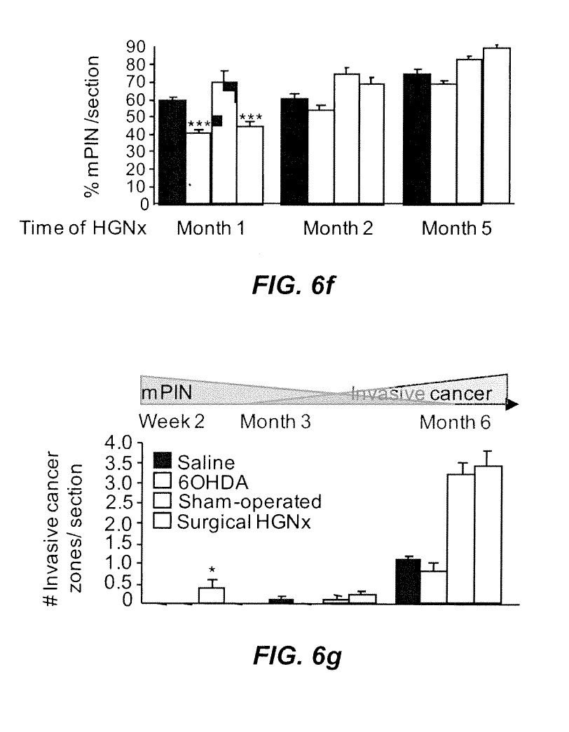

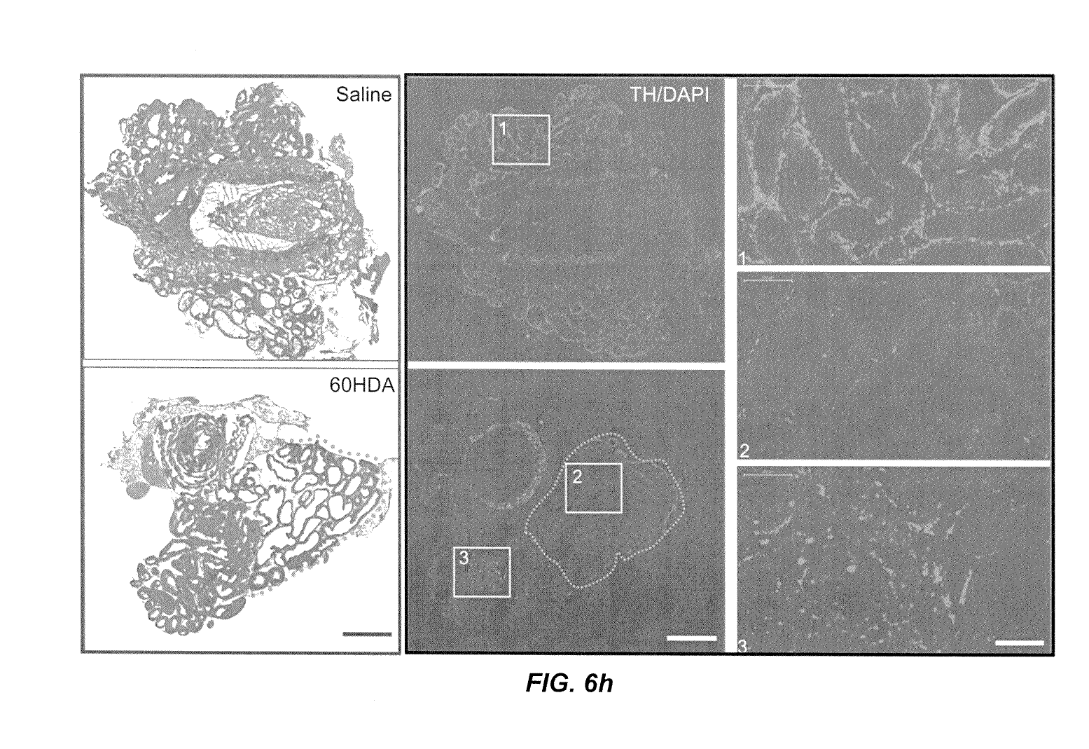

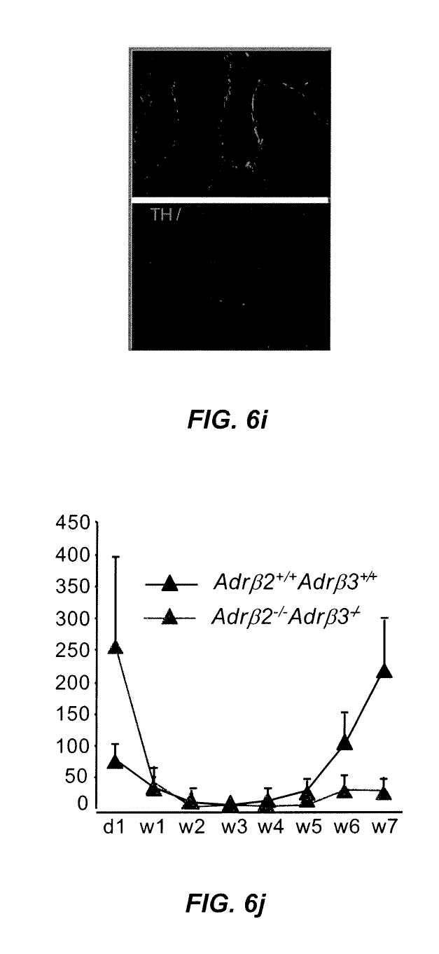

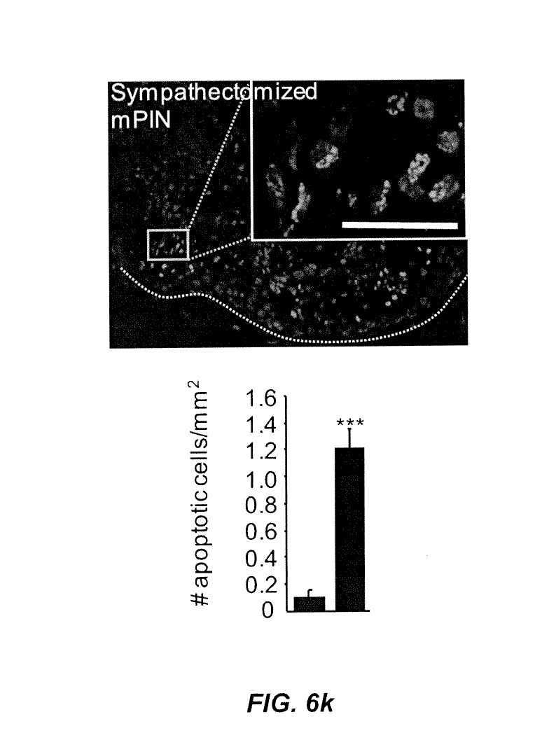

Adrenergic nerve fibers were lesioned using 6-hydroxydopamine (6OHDA) injections alone, or prior to administering G-CSF in animals previously grafted orthotopically with PC-3luc cells, to assess the functional role of nerves in tumors. 6OHDA treatment specifically destroyed TH+neural fibers expressing NF-L located in the basal neural layer underneath the prostate epithelium (FIG. 5a, b), without affecting NF-L and NF-H positive fibers (parasympathetic) surrounding epithelial cells (FIG. 5a, b). Unexpectedly, bioluminescence analyses of orthotopic tumors and TUNEL assays revealed that 6OHDA-induced sympathectomy dramatically prevented the development of, i.e., reduced cancer cell survival in, the primary tumors, indicating an important role for sympathetic neural activity in tumor initiation (FIG. 2i, FIG. 5c, d). 6OHDA also triggered in vivo apoptosis of epithelial cells in the healthy prostate (FIG. 5c, d), whereas it had no direct cytotoxicity on tumor cells in vitro (FIG. 5f), but triggered apoptosis of prostate epithelial cells in vivo (FIG. 5e). Consequently, the primary site deprived of live tumor cells did not yield any detectable metastases (FIG. 2j). To ascertain whether sympathetic signals were locally delivered in the tumor microenvironment, surgical microsection of hypogastric nerves that carry sympathetic fibers into the prostate was carried out (FIG. 6a). Immunofluorescence staining for TH+ fibers confirmed sympathectomy. Surgical denervation or sympathectomy dramatically inhibited tumor development within three weeks after orthotopic injection of PC-3luc tumor cells, whereas sham-operated animals developed exponentially from week 5. These data indicate that SNS signals regulate prostate tumor cell development and tumor initiation.

Immunodeficient nude (nu/nu) mice were crossed with animals deficient in either β2, β3, or both adrenergic receptors to study the role of these mediators of SNS signaling in prostate cancer development (tumor initiation). (These adrenergic receptors had previously been shown to transducer SNS signals in the bone marrow niche.) Remarkably, injection of PC-3luc cells into the prostate of nu/nu Adrβ2^{-/-} Adrβ3^{-/-} mice led, within 24 hours, to a significant reduction in tumor cell survival as determined by bioluminescent signal readings (FIG. 6b). Whereas engraftment appeared slightly delayed in nu/nu mice lacking a single adrenergic receptor, it was severely compromised in nu/nu Adrβ2^{-/-} Adrβ3^{-/-} animals (FIG. 6b). Tumor cell dissemination into the lymph nodes and distant organs was significantly reduced, owing to, at least in part, the marked reduction of live cells at the primary site (FIG. 6c, d). These observations did not depend on the prostate cancer cell line because tumor development was also impaired when LNCaP prostate tumor cells stably expressing luciferase were orthotopically injected in nu/nu Adrβ2^{-/-} Adrβ3^{-/-} animals (FIG. 6j). These data indicate an important role for both Adrβ2 and Adrβ3 in initiating tumor development.

To ascertain the role of sympathetic innervation in tumor initiation using a genetic model, the effect of chemical or surgical sympathectomy on prostate cancer in cMyc mice was evaluated. For this study, transgenic cMyc mice expressing human c-Myc under the ARR2/probasin promoter expressed specifically in the prostate (AAR2/Pbsn-MYC) were used. This transgenic model leads to the complete penetrance of mouse prostatic intraepithelial neoplasia (mPIN) from postnatal week 2, progressing to invasive adenocarcinomas between 3 to 6 months of age (FIG. 6e). To this end, mice were chemically sympathectomized one month prior to histological analyses. Sympathectomy, e.g., chemical sympathectomy, at an early age (i.e., one month after birth) was found to significantly reduce the frequency of mPIN, as determined in analyses of tissue sections and increased apoptotic rates of cMyc neoplastic cells (FIG. 6f, g, k). In contrast, the prevalence of mPIN and invasive cancer zones were not significantly altered when the sympathectomy was performed at later stages of tumor progression (i.e., two months or more after birth; FIG. 6f, g). In addition, immunostaining of prostate sections from cMyc mice showed that mPIN and cancer zones were highly innervated by a dense network of TH+ fibers, as compared to normal tissues (FIG. 6h, Box 1). In sharp contrast, in 6OHDA-lesioned mice, large areas of normal-appearing acini that exhibited only faint TH+ staining were observed (FIG. 6h, Box 2). Areas affected by mPIN also stained strongly for TH (FIG. 6h, Box 3), indicating that 6OHDA likely denervated heterogeneously, and further supporting the relationship between the presence of SNS fibers and cancer initiation.

The data disclosed in this Example establish that sympathetic nerve innervation accompanies tumor initiation. Consequently, inhibition of SNS innervation would be expected to interfere with tumor initiation, providing an effective prevention and/or treatment of cancer. Accordingly, the disclosure provides a method of inhibiting cancer initiation in a tissue of a subject comprises administering a prophylactically or therapeutically effective amount of a sympathetic nervous system inhibitor. In some embodiments, the tissue is prostate. In some embodiments, the subject is a human.

### Example 4

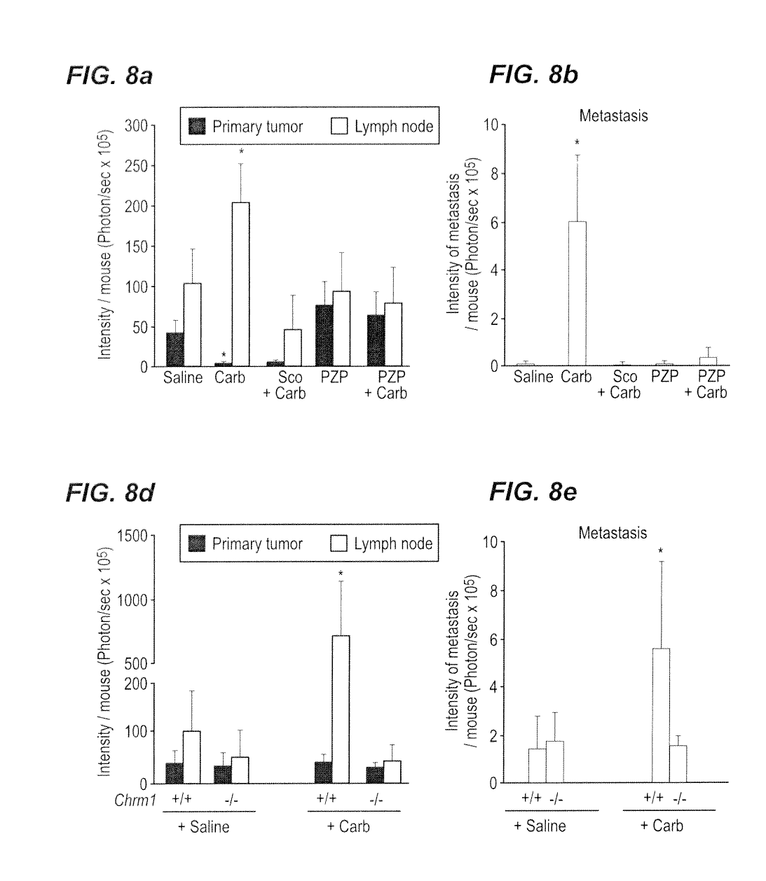

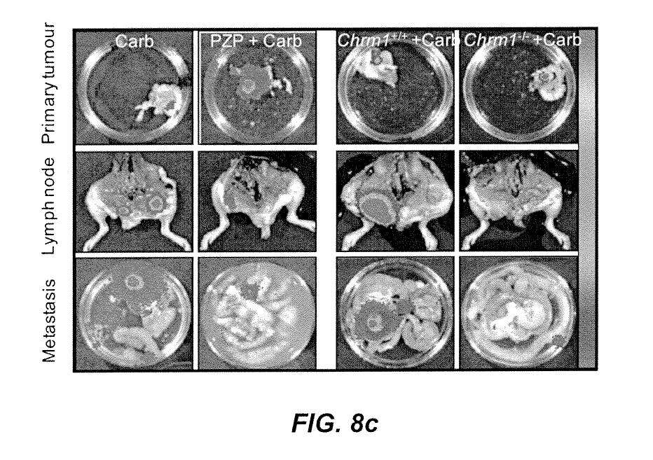

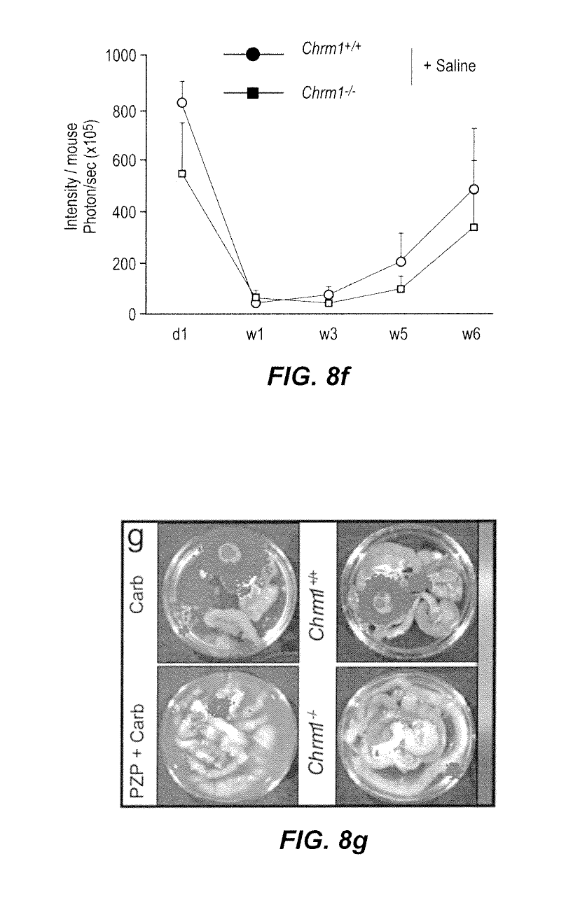

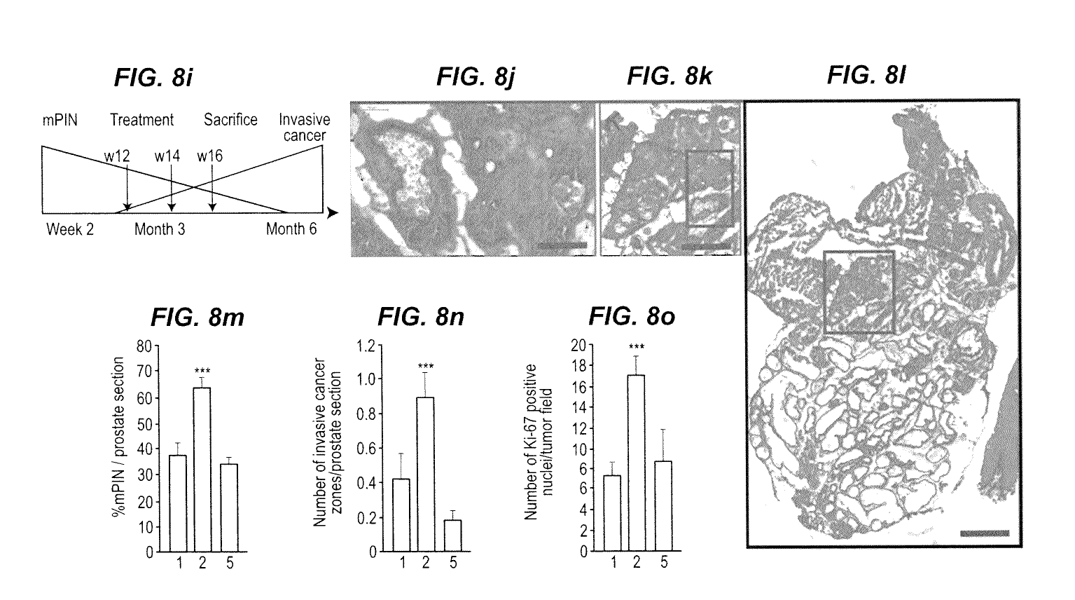

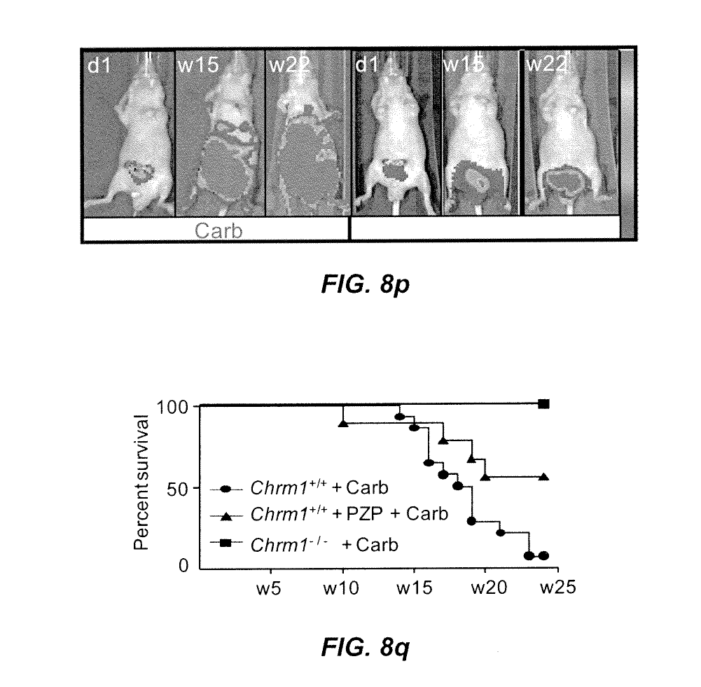

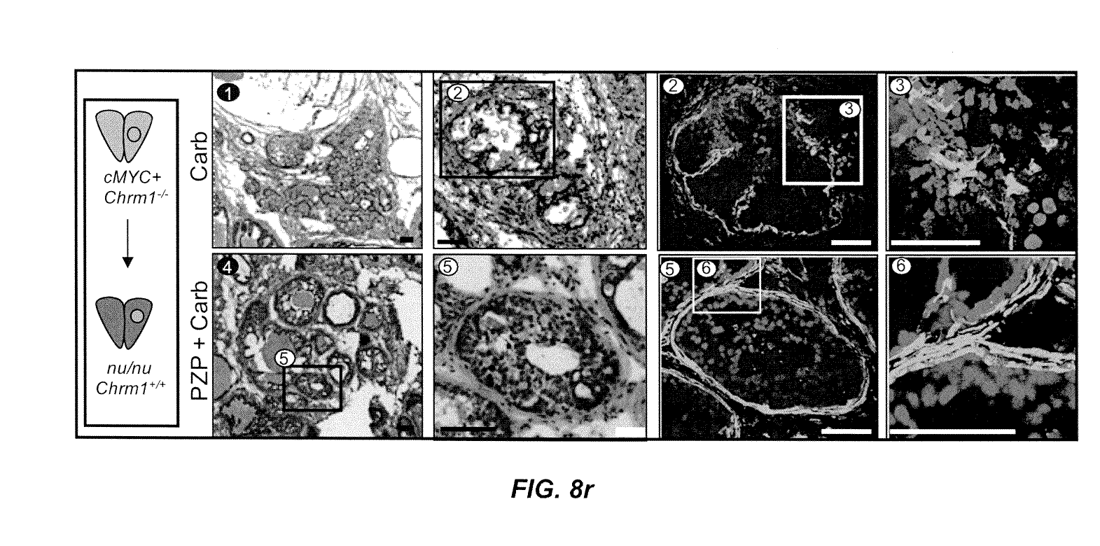

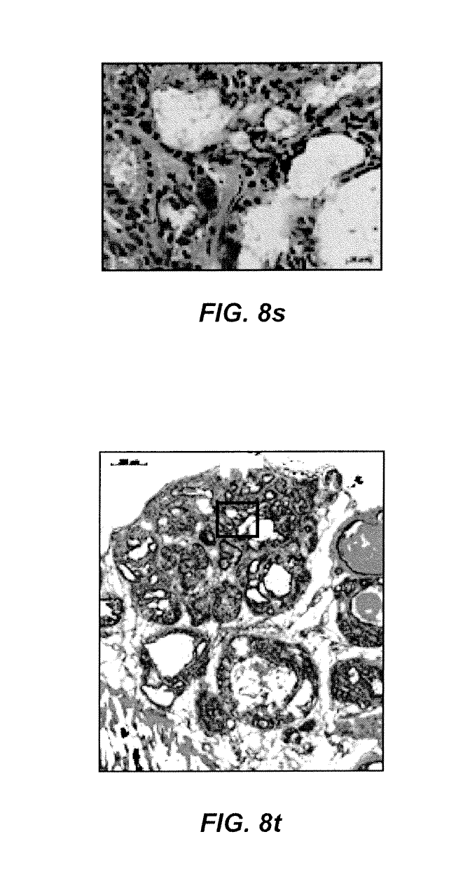

Several homeostatic functions of mammals require finely regulated, balanced signals from both the sympathetic and parasympathetic nervous systems (PNS). In initial studies, it was noted that G-CSF administration protected TH+ fibers against 6OHDA-induced damage and tumor engraftment (primary tumor development) was restored (FIG. 2i, FIG. 4a, b). These tumors, however, were not capable of producing metastases (FIG. 2j). Unexpectedly, immunofluorescence analyses revealed that G-CSF selectively promoted the infiltration of VAChT+ parasympathetic neo-nerves in that G-CSF-induced neo-fibers were largely VAChT+ parasympathetic nerves (FIG. 2k, l), and in that 6OHDA-induced sympathectomy also altered G-CSF-induced generation of VAChT+ parasympathetic neo-fibers both in tumors (FIG. 2k, l) and in the normal, tumor-free prostate gland (FIG. 4a, b). Stated in the alternative, the outgrowth of the VAChT+ fibers is compromised in G-CSF-treated sympathectomized mice (FIG. 2k, l; FIG. 8a, b). Thus, these results confirm that adrenergic fibers are important for tumor initiation, and that the two branches of the autonomic nervous system are functionally linked. In addition, the correlation of parasympathetic fiber density and the development of VAChT+ neo-fibers and cancer dissemination indicates a role for cholinergic nerves and their neurotransmitter's receptors.

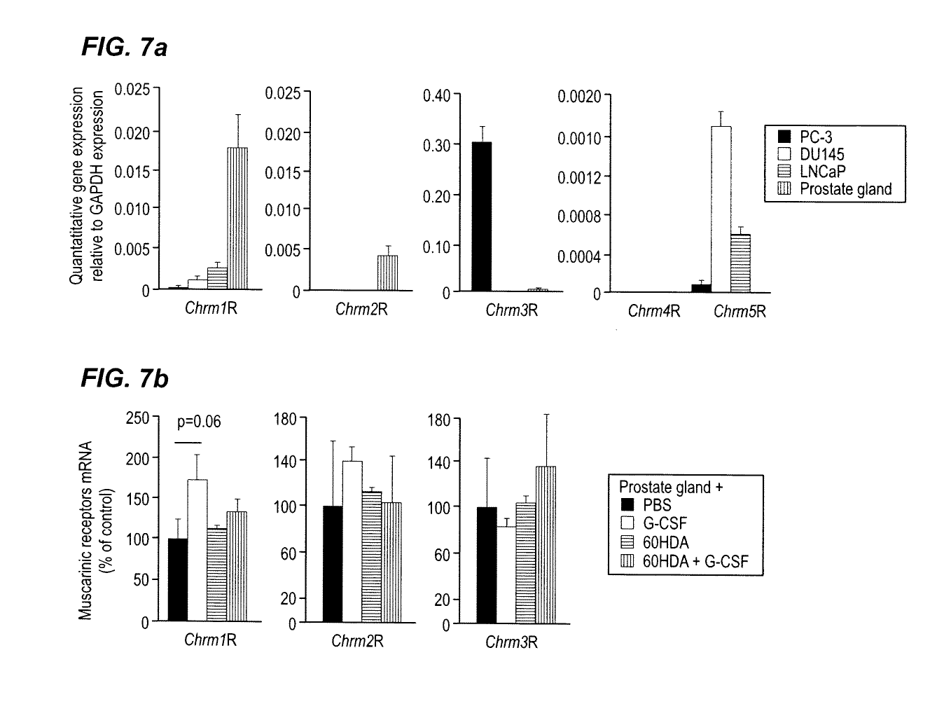

In vitro studies with prostate cancer cell lines have shown an effect of exogenous acetylcholine (Ach) on cell proliferation through M3R, a receptor that is highly expressed by some tumor epithelial cells. Attention was drawn to postganglionic parasympathetic nervous system (PNS) neurons, which activate muscarinic cholinergic receptors on the effector organ. This led to a focus on muscarinic receptor expression. Expression profiles of the five known muscarinic receptor genes in the mouse prostate gland and prostate cancer cell lines were determined. PC-3 cells largely express Chrm3 (M3R), whereas Chrm5 (M5R) was the predominant receptor in DU145 and LNCaP cells (FIG. 7a). Acetylcholine was previously reported to induce prostate cancer cell line proliferation in vitro via Chrm3. In contrast, consistent with prior studies in human prostate tissues, Chrm1 (M1R) was expressed at high levels in the healthy prostate gland (FIG. 7a, b). Based on these results, it was hypothesized that parasympathetic activity operates through Chrm1 (M1R) expressed in the prostate tumor microenvironment.

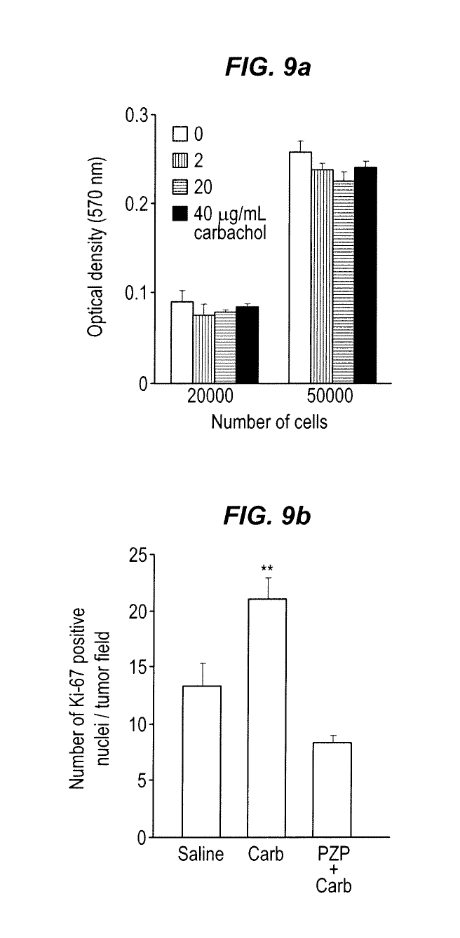

To investigate this issue, PC-3luc-grafted nude mice were treated with the non-selective parasympathomimetic carbamoylcholine chloride (carbachol) and the role of cognate muscarinic receptors was tested using pharmacological antagonists. Carbachol treatment significantly enhanced the invasion of inguinal lymph nodes that drain the prostate gland (FIG. 8a, c). The invasion was mediated by a muscarinic receptor since it was inhibited by a non-selective muscarinic antagonist (scopolamine; FIG. 8a, b). Furthermore, specific blockade of Chrm1 (M1R) using pirenzepine also inhibited lymph node invasion and metastasis (FIG. 8a, c). To assess whether the invasion of lymph nodes was associated with metastasis to distant organs, the bioluminescence contained in each organ was quantified ex vivo. Treatment with carbachol dramatically increased (about 6-fold) tumor cell dissemination compared to the control group (FIG. 8b, g). Furthermore, distant metastases were inhibited by the blockade of Chrm1 (M1R) using either scopolamine or pirenzepine injections. Treatment of PC-3luc cells with carbachol did not induce any proliferation in vitro, consistent with results in vivo (FIG. 9a, b), indicating that the effect of the parasympathomimetic drug is mediated through the microenvironment and indicating that the effect was not tumor-cell autonomous. Vascular hemodynamics were not altered in carbachol-treated mice (see Example 8 and Table 2, below), indicating that the observed effects were not due to a non-specific cardiovascular dysfunction. Thus, these results indicate that cholinergic agonistic activity may promote lymph node invasion and metastasis by acting on the tumor stroma. Accordingly, the disclosure provides a method of treating cancer in a subject by administering a therapeutically effective amount of an inhibitor of the parasympathetic nervous system. In some embodiments, the cancer treatment comprises inhibition of metastasis. In some embodiments, the subject being treated is a human or a non-human animal, such as a non-human mammal. In some embodiments, the inhibitor is a Chrm1 inhibitor, carbamoylcholine (carbachol) or pirenzepine (PZP).

### Example 5

An experiment was designed to assess whether the parasympathetic nervous system influence on tumor cell development and egress operated via the Chrm1 (M1R) muscarinic receptor.

### Animal Procedures

For cholinergic experiments, 15 days after tumor cell implantation (2×10⁵ PC-3luc cells), animals received carbamoylcholine chloride (Sigma-Aldrich) at 250 (day 0), 300 (day 1), 350 (day 2), and 500 μg/kg/day (day 4) (every 12 hours, 8 divided doses, i.p., in saline) alone or in combination with scopolamine hydrobromide (Sigma, 1 mg/kg) or pirenzepine dihydrochloride (Sigma, 6 mg/kg). A second cycle was administered at week four and mice were sacrificed one week later (five weeks after graft). For cholinergic experiments using the nu/nu Chrm1^{tm1Stl+/+} and nu/nu Chrm1^{tm1Stl-/-} animals, mice were sacrificed at week six post-graft. For the survival study, grafted mice were treated as described hereinabove from week 4, every two weeks for up to 25 weeks or until death. Disease progression was monitored by bioluminescence scanner. For the c-Myc model, three-month-old mice were injected with Carbachol alone or in combination with pirenzepine for four days following the protocol described above. One week later, mice received a second round of treatment and were then sacrificed at month four. For cMyc+ acini implantation, two-month-old cMyc+ Chrm1^{tm1Stl-/-} acini were implanted into six-week-old nu/nu Chrm1^{tm1Stl+/+} recipients. After 5 weeks, mice were treated with two cycles (week 6 and 8) of carbamoylcholine chloride alone or combined with pirenzepine dihydrochloride as described above. Mice were sacrificed at week 10.

### Results

To determine definitively whether tumor parasympathetic cholinergic signals target Chrm1 (M1R) receptors in the stroma, M1R-deficient (Chrm1)^{-/-} mice were crossed with nu/nu animals and PC-3luc cells were implanted orthotopically in the resulting nu/nu Chrm1^{-/-} and nu/nu Chrm1^{+/+} control animals. Carbachol-induced tumor cell egress and metastasis were dramatically reduced when the prostate microenvironment was deprived of Chrm1 (M1R) expression (FIG. 8d-g). Notably, deficiency in Chrm1 (M1R) signaling did not impact primary tumor development in the prostate because the primary tumor growth curve of Chrm1^{-/-} mice paralleled that of control Chrm1^{+/+} mice (FIG. 8h). Importantly, cholinergic-induced metastatic tumors and survival were dramatically improved by pharmacologic blockade or genetic disruption of stromal Chrm1 (FIG. 8p, q).

To confirm further the effect of cholinergic agonists on tumor cell spreading, cMyc transgenic mice were evaluated (FIG. 8i-l). The transgenic cMyc mice were also bred with Chrm1^{-/-} animals (FIG. 8i-o). Injections of carbachol into three-month-old transgenic mice significantly increased the incidence of mPIN (FIG. 8m) and accelerated the transition of these neoplastic lesions to invasive carcinoma at four months of age (FIG. 8n). In addition, carbachol treatment significantly increased the proliferative index of tumor cells (FIG. 8o). Treatment of cMyc mice with pirenzepine or genetic deletion of Chrm1 completely inhibited carbachol-induced malignancy progression (FIG. 8m-o). cMyc+ Chrm1^{-/-} prostate acini were also implanted into the dorsal lobe of the prostate of healthy nude mice. In the resulting chimeric prostate, only the donor prostate tissue can develop cancer and only the recipient tissue can respond to Chrm1-mediated signals. Treatment of engrafted mice with carbachol switched the tumor behavior to an invading phenotype with stromal Chrm1-mediated disruption of the basement membrane and proliferation of tumor epithelial cells (FIG. 8r). Taken together, the studies in two models of prostate cancer demonstrate that cholinergic signals transduced in the tumor stroma by the type-1 muscarinic receptor promote prostate cancer invasion and dissemination.

### Example 6

The disclosure provides experimental evidence in the form of data establishing that the materials and methods are effective not only in mice, a recognized human cancer model, but in humans as well.

### Human Prostate Samples

Prostate samples were harvested from radical prostatectomy specimens. Patients with Gleason 3+4 adenocarcinoma in 50% of the gland (pT3aN0, pT3bN0, and pT2cN0) provided the samples. After extirpation, tissues were immediately immersed in OCT medium (Tissue Teck®, Sakura Finetek, Torrance, Calif.) and frozen. In some experiments, five-micrometer sections were used for staining.

### Results

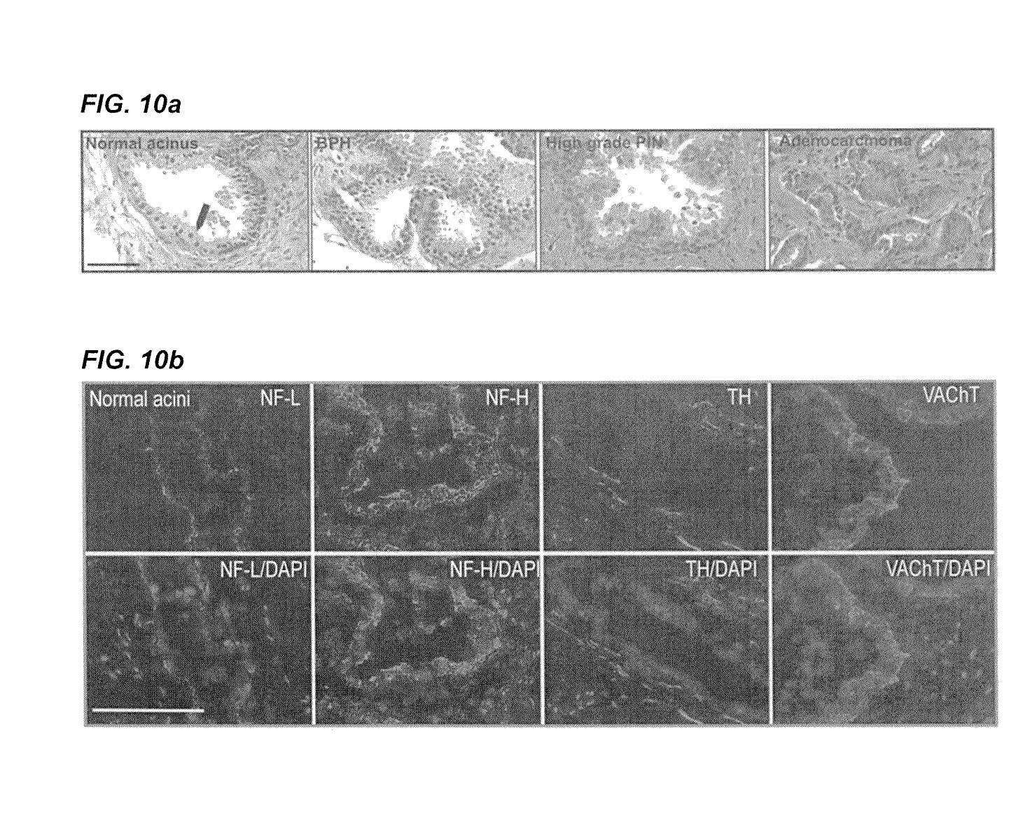

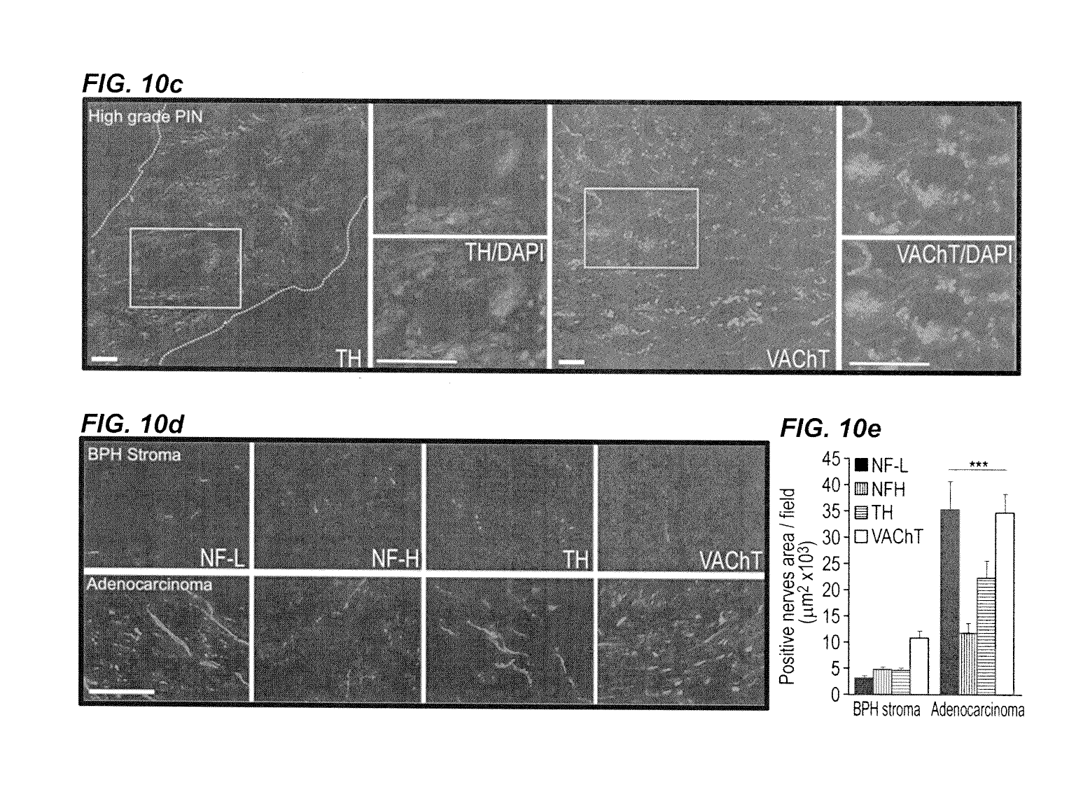

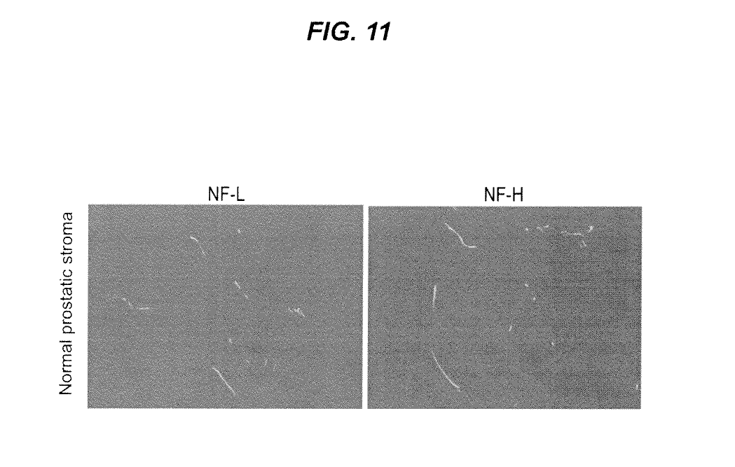

Healthy human tissues, benign prostatic hyperplasia (BPH) biopsies, and tumor sections from patients harboring invasive prostate cancer were stained (FIG. 10a). Normal prostate epithelium was predominantly innervated by thin, well-organized, mature cholinergic fibers (NF-H+/VAChT+) and by some sparse NF-L+ nerves, running along the prostate acini and surrounding epithelial cells (FIG. 10b). Notably, very few TH+ fibers were detected in the normal prostate epithelium. However, regions of high grade PIN exhibited strong VAChT+ fiber arborization throughout the stroma and closely surrounding proliferative neoplastic epithelial cells (FIG. 10c). In addition, the distribution of adrenergic nerves was also altered with the patchy recruitment of TH+ fibers around tumor epithelial cells (FIG. 10c). In sharp contrast, sections from adenocarcinomas displayed dense and disorganized bundles of immature (largely NF-L+) neo-fibers (FIG. 10d). Overall, densities for both parasympathetic (VAChT+) and sympathetic (TH+) autonomic fibers were much greater in human prostate adenocarcinoma as compared to BPH tissues (FIG. 10e). Taken together, these data reveal the recruitment of neo-fibers in tumor tissues and indicate a putative tumor neurogenic switch for progression to invasive cancer.

### Example 7

A variety of therapeutic methods are provided by the disclosure, in which therapeutically and/or prophylactically effective amounts of an adrenergic receptor antagonist are useful in inhibiting or preventing tumor initiation and in inhibiting tumor development, and wherein a cholinergic receptor (e.g., type 1) antagonist is useful in inhibiting or preventing tumor invasion and spreading (i.e., metastasis), and wherein each of these anti-cancer therapeutics is synergistically combined with one or more conventional anti-cancer therapies, such as a chemotherapy or surgery, to yield synergistic effects in treating cancer.

### Antagonists of β2 and β3 Adrenergic Receptors

β2 and β3 adrenergic receptors control tumor initiation and early stages of cancer development. Prophylactic use of β2/β3 antagonists are expected to prevent the occurrence, and/or relapse after complete response, of prostate adenocarcinoma. This treatment is initiated with consideration given to patient history, family history, cancer risks in individuals tested for genetic mutations, and other conventional factors routinely considered when one of skill is contemplating anti-cancer treatment of a human or other animal such as a non-human mammal. Most non-selective beta-adrenergic antagonists (e.g., propranolol) do not inhibit the β3 adrenoreceptor. A recent clinical study using non-selective β2/β1 beta blockers in breast cancer, however, showed a clinical benefit by reducing mortality (Barron et al, J. Clin. Oncol. 33:5422 (2011)). This study highlights the fact that selective activity on β2 adrenoreceptor regulates tumor development. By contrast, administration of selective β1 beta blockers does not display any clinical benefits.

Drugs that affect the development of the sympathetic nervous system (e.g., anti-NGF, anti-GDNF) are combined with selective beta blockers to potentiate their therapeutic effect. Accordingly, the disclosure provides a method for treating or preventing a cancer, or inhibiting metastasis thereof, comprising administering an antagonist of the sympathetic nervous system and an antagonist of the β2 adrenoreceptor in a combined amount effective to treat or prevent the cancer, thereby treating or preventing, or inhibiting the metastasis of, the cancer. Any of the cancers, sympathetic nervous system antagonists or β2 adrenoreceptor antagonists disclosed herein are contemplated for use in the method, including the use of anti-NGF antibody and anti-GDNF antibody as antagonists of the sympathetic nervous system. For drugs and metabolic pathways that have an opposite effect, see International Patent Application No. PCT/US2011/051640, incorporated by reference herein in its entirety.

### Antagonists of Chrm1 Muscarinic Receptors

The Chrm1 muscarinic pathway promotes invasion and tumor spreading in lymph nodes and distant sites. Thus, drugs that inhibit this pathway are expected to be useful in adjuvant settings. For example, administration of Chrm1 pathway inhibitors prior to surgery is expected to prevent local invasion of the prostate gland and reduce distant metastases occurring perioperatively by surgical manipulation of the tumor. Perioperative drug combinations contemplated herein include β2/β3 antagonists with Chrm1 inhibitors in order to avoid any local engraftment of tumor cells left in margins of the primary organ. Thus, the disclosure provides a method of inhibiting tumor engraftment by administering a therapeutically effective amount of a Chrm1 pathway inhibitor to a human patient or a non-human animal such as a non-human mammal, thereby inhibiting tumor engraftment. Additionally, the disclosure provides a method of inhibiting tumor metastasis by administering a therapeutically effective amount of a Chrm1 pathway inhibitor to a human patient or a non-human animal, such as a non-human mammal. In these methods, a therapeutically effective amount includes a prophylactically effective amount.

Selective antagonism of stromal expression of the type 1 muscarinic receptor is expected to synergize with cancer chemotherapies, such as alkylating drugs, antimetabolites, antibiotics and hormonal agents, each of which impairs mitosis, selectively affecting fast-dividing tumor cells. Accordingly, the disclosure provides a method of treating or preventing cancer, or preventing metastasis thereof, comprising administering a therapeutically effective amount of a type 1 muscarinic receptor antagonist in combination with administration of any known anti-cancer treatment, including but not limited to cancer chemotherapies, such as alkylating drugs, antimetabolites, antibiotics and hormonal agents, and cancer physical therapies, including but not limited to surgery to destroy or remove part or all of a cancer tissue.

Combination of Anti-Angiogenic Drugs with β2 Antagonists and/or Chrm1 Muscarinic Antagonist

Anti-VEGF therapies can elicit tumor resistance and progression to invasiveness with increased number of distant metastases (Paez-Ribes et al., Cancer Cell 15:220 (2009); Ebos et al., Cancer Cell 15:232 (2009)). The tumor does not develop in the absence of adrenergic signals, indicating that there is crosstalk between the development of neo-nerves and new vessels in the tumor. Thus, the combination of anti-angiogenic drugs and beta-adrenergic antagonists is expected to have a synergistic effect on tumor development and the combination therapies are also expected to overcome resistance to anti-angiogenic drugs.

Cholinergic signals trigger tumor dissemination towards the periphery. This suggests that intra-tumor vessels might respond to parasympathetic tumor innervation by vasodilatation or permeabilization. Combination of antagonists of the Chrm1 receptor with anti-angiogenic drugs could impair the development of tumor neo-vascularization and peripheral dissemination of tumor cells.

In the disclosures provided herein, several distinct functions for the two branches of the autonomic nervous system in tumor initiation and progression toward metastasis have been identified (FIG. 1). These results establish, longitudinally, the early events leading to metastasis from an experimental orthotopic tumor model. Whether prostate tumors were generated in a xenogeneic model, or arose spontaneously either in oncogene-driven transgenic mice or in primary human cancer, recruitment by cancerous tissue of a dense network of autonomic nerves has been observed. Perineural invasion was shown in clinical studies to confer a poor prognosis in prostate cancer and other carcinomas, presumably by providing a gateway toward hematogenous spread. The experimental results disclosed herein indicate that a reverse phenomenon occurs wherein the prostate tumor itself is invaded by nerves that, in turn, regulate cancer initiation and progression. Thus disclosed herein are the dual, complementary functions for autonomic innervation in tumors such as prostate tumors, where adrenergic fibers from the SNS are critically required for regulating tumour initiation through stromal β2 and β3 adrenergic receptors, and where cholinergic fibers of the PNS drive tumor cell invasion, migration and distant metastases through stromal Chrm1 expression.

### Example 8

### Assessment of Hemodynamics and Cardiac Function

To evaluate the effect of a cholinergic agonist on blood flow, mice were anesthetized and prepared for intravital microscopy as described (Hidalgo et al., Nat. Med. 15:384-91 (2009), incorporated herein by reference in its entirety) one hour after carbachol injection. Centerline red blood cell velocities were measured for 10 venules and 10 arterioles in two independent experiments. Wall shear rates (γ) were calculated based on Poiseuille's law for a Newtonian fluid, γ=2.12 (8Vₘₑₐₙ)/Dv or Da, where Dv and Da are the venular or arterial diameters, the mean blood flow velocity (Vₘₑₐₙ) was estimated as V_{RBC}/1.6, and 2.12 is a median empirical mean, correction factor obtained from actual velocity profiles measured in microvessels in vivo. The blood flow rate was calculated from the formula: Vₘₑₐₙ π D²/4.

Cardiac function was assessed by echocardiography using the Vevo 2100 ultrasound imaging system where mice were treated for four consecutive days with saline or carbachol according to the protocol described above (n=4/group). For imaging, animals were anesthetized with a mixture of O₂/1.5% isoflurane and then positioned ventral side up on the platform of the imaging system. ECG signal and respiratory rate were captured through the electrode pads on the advanced physiological monitoring unit and transmitted to the Vevo system for monitoring. Cardiac examinations were performed in two dimensional images using the parasternal long axis (PLAX) view in B-Mode with a 1MS550D 40 MHz probe. Two cineloops (300 frames/cineloop) were recorded per animal and then analyzed on two diastoles and two systoles per animal. The endocardial stroke volume and endocardial diastolic volume were determined to calculate the ejection fraction.

As noted herein, Vascular hemodynamics were not altered in carbachol-treated mice (Table 2), indicating that the observed effects were not due to a non-specific cardio-vascular dysfunction. Thus, these results indicate that cholinergic agonistic activity promotes lymph node invasion and metastasis by acting on the tumor stroma.

TABLE 2
Hemodynamic measurements after carbachol administration

| | | | Control | Carbachol | P value |
|---|---|---|---|---|---|
| Intravital microscopy | | n = 10 vessels | | | |
| Venules | | | | | |
| Diameter (μm) | | | 35.5 ± 1.2 | 33.7 ± 1.3 | 0.45 |
| Blood Flow rate (nl/sec) | Vₘₑₐₙ × π × d²/4 | | 821 ± 93 | 863 ± 128 | 0.25 |
| Wall shear rate (g) | 2.12 (8 Vₘₑₐₙ)/d | | 397 ± 38 | 469 ± 59 | 0.15 |
| RBC velocity (mm/sec) | | | 1.30 ± 0.10 | 1.48 ± 0.19 | 0.19 |
| Arterioles | | | | | |
| Diameter (μm) | | | 38.3 ± 1.2 | 38.1 ±0.8 | 0.45 |
| Blood Flow rate (nl/sec) | Vₘₑₐₙ × π × d²/4 | | 1518 ± 110 | 1737 ± 148 | 0.08 |
| Wall shear rate (g) | 2.12 (8 Vₘₑₐₙ)/d | | 579 ± 27 | 698 ± 69 | 0.05 |
| RBC velocity (mm/sec) | | | 2.06 ± 0.11 | 2.47 ± 0.22 | 0.09 |
| Echocardiography | | n = 4 mice | | | |
| Ejection fraction (%) | (Endocardial SV/Endocardial | | 63 ± 8 | 65 ± 4 | 0.85 |
| | Vol; d) × 100 | | | | |
| Endocardial volume, | (4π/3) × (End Major; d/2) × {End | | 35.7 ± 7.9 | 34.4 ± 7.9 | 0.87 |
| diastole (μl) | Area; d/(π(End Major; d/2))}2 | | | | |
| Endocardial volume, | (4π/3) × (End Major; s/2) × | | 14.8 ± 5.3 | 11.7 ± 3.0 | 0.51 |
| systole (μl) | {EndArea; s/(π(End | | | | |
| | Major; s/2))}2 | | | | |

Vₘₑₐₙ, Mean blood flow velocity; d, diameter; SV, Stroke Volume which is calculated by substraction of the left ventricle end-systolic volume from the left ventricle end-diastolic volume; Vol, volume; End Major, endocardial major represent the maximal ventricular length; EndArea, endocardial area; s, systole; d, diastole.

The disclosed subject matter has been described with reference to various specific and preferred embodiments and techniques. It should be understood, however, that many variations and modifications may be made while remaining within the spirit and scope of the disclosed subject matter.

1-38. (canceled)

39. A method of inhibiting tumor metastasis in a patient comprising administering to a patient a M1 muscarinic receptor inhibitor in an amount effective to inhibit tumor metastasis in a patient.

40. The method of claim 39, further comprising administering an amount of a β adrenergic receptor inhibitor effective to inhibit tumor initiation.

41. The method of claim 40, wherein the adrenergic receptor inhibitor is a β2 adrenergic receptor inhibitor, a β3 adrenergic receptor inhibitor, or both a β2 adrenergic receptor inhibitor and a β3 adrenergic receptor inhibitor.

42. The method of claim 41, wherein the β2 adrenergic receptor inhibitor is selected from the group consisting of an anti-β2 antibody, butaxamine, propranolol and ICT 118,551.

43. The method of claim 41, wherein the β3 adrenergic receptor inhibitor is selected from the group consisting of an anti-β3 antibody and SR 59230A.

44. The method of claim 39, wherein the inhibitor is selected from the group consisting of an anti-M1 receptor antibody, scopolamine, pirenzepine, atropine, dicycloverine, tolterodine, oxybutynin, ipratropium, mamba toxin MT7, solifenacine, procyclidine, mebeverine, benzatropine, cyclopentolate, trihexyphenidyl/benzhexol, tiotropium, flavoxate, dicyclomine, dimenhydrinate, diphenidramine, tropicamide and telenzepine.

45. The method of claim 39, wherein the patient has a cancer selected from the group consisting of prostate cancer, breast cancer, melanoma, gastric cancer, colon cancer, liver cancer, pancreatic cancer, esophageal cancer, lung cancer and urogenital cancer.

46. The method of claim 39, wherein the inhibitor is administered at a daily dosage of 10 μg/kg to 10 mg/kg.

47. A method of diagnosing a cancer in a patient comprising assaying a tissue sample from the patient for a marker for neurofilament-L and for a marker for neurofilament H, wherein presence of a marker for neurofilament-L and absence of a marker for neurofilament H is indicative of cancer.

48. The method of claim 47, wherein the tissue is prostate tissue.
